(19) United States
(12) Patent Application Publication
(10) Pub. No.: US 2003/0185851 A1
(43) Pub. Date: Oct. 2, 2003

(54) Tet transactivator system

(76) Inventors: Dominique Soldati, London (GB); Markus Meissner, London (GB)

Correspondence Address: Jonathan P. Osha, ROSENTHAL & OSHA L.L.P., Suite 2800, 1221 McKinney Street, Houston, TX, 77010, US

(21) Appl. No.: 10/102,143

(22) Filed: Mar. 20, 2002

(51) Int. Cl. A61K 39/002; C07H 21/04; C12P 21/02; C12N 9/22; C12N 1/10; C12N 15/74

(52) U.S. Cl. 424/191.1; 435/69.3; 435/199; 435/258.1; 435/320.1; 536/23.7

(57) ABSTRACT

A transcriptional activator of T. gondii is provided which comprises the tetracycline repressor (TetR) operatively linked to a transacting factor of T. gondii. Strains of T. gondii transformed with a vector containing such a transactivator may be used to prepare vaccine compositions or to identify essential genes in the parasite. The system provided may be useful in other Apicomplexan species such as Plasmodium falciparum, Plasmodium vivax, Plasmodium berghei, Plasmodium yoelii, Plasmodium knowlesi, Trypanosoma brucei, Entamoeba histolytica, and Giardia lambia.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF INVENTION

1. Field of the Invention

The present invention relates to nucleic acid constructs that can act as inducible transactivator systems in Apicomplexan parasites which can be used to create attenuated strains of the parasites that can act as vaccines to protect against infection by wild-type parasite. The transactivator system also permits the systematic study of the genes in Apicomplexan parasites.

2. Background Art

An inducible control of individual gene expression is a prerequisite to study the function of essential genes. Several strategies and tools associated with DNA transformation have been developed in the human and animal pathogens of Toxoplasma gondii and the Plasmodium species. The currently available methods to study essential gene function are antisense RNA and ribozyme technology (Nakaar et al J. Biol. Chem. 274 5083-5087 (1999); Gardiner et al Mol. Biochem. Parasitol 110 33-41)). The development of a controlled gene expression system would not only permit the generation of conditional knockouts but would also allow the study of mutated forms of endogenous genes and the expression of toxic genes.

In the originally described tetracycline-controlled inducible expression system (Gossen, M. & Bujard, H., Proc. Nat'l Acad. Sci. USA 89 5547-5551 (1992), the fusion of the tetracycline repressor (TetR) with the activating domain of the Herpes simplex virion protein 16 (VP16) has converted the repressor into an efficient tetracycline-controlled transactivator (tTA). In that case, a minimal promoter fused to tetracycline operator (tetO) sequences is activated in cells expressing tTA and becomes silent in the presence of tetracycline. This system is highly efficient in regulating genes in diverse eukaryotic organisms but has not been established in any protozoan parasite

In contrast, the TetR system regulates gene expression in a number of protozoan parasites, including Trypanosoma brucei (Wirtz, E. & Clayton, C., Science 268 1179-1183 (1995)), Entamoeba histolytica (Hamann et al Mol. Biochem. Parasitol. 84 83-91 (1997)), Giardia lambia (Sun, C. H. & Tai, J. H., Mol. Biochem. Parasitol. 105 51-60 (2000)), and Toxoplasma gondii (Meissner et al Nucleic Acids Res. 29 e115 (2001). As in bacteria, TetR interferes with initiation of transcription by binding to tetO sequences, placed properly in the vicinity of the promoter region of protozoan genes. In the presence of tetracycline the repressor ceases to bind to the tetO sequence and thus interference is abolished, rendering the promoter active.

T. gondii exhibits a remarkably high frequency of stable transformation coupled to the preferential integration at random throughout the genome which have previously been exploited to design insertional mutagenesis strategies leading to the cloning of non-essential genes and to the identification of developmentally regulated genes by promoter trapping (Donald et al J. Biol. Chem. 271 14010-14019 (1996); Knoll, L. J., & Boothroyd, J. C., Mol. Cell. Biol. 18 807-814 (1998)).

An inducible system based on the tet-Repressor has been reported to control gene expression in several protozoan parasites but best optimised and applied predominantly in Trypanosoma brucei. Indeed, the existence of trans-splicing in kinetoplastida offers a unique opportunity to combine this tetracycline-dependent repression with the powerful T7 polymerase transcription (Wirtz et al Mol. Biochem. Parasitol. 99 89-101 (1999)). In contrast, the broadly used and tighter transactivator system, (tTA) composed of tetR-VP16 fusion has not been reported to function in any protozoan parasites. Recent studies to investigate the use of the tet-Repressor to control gene expression in T. gondii found that the tTA was totally inactive (Meissner et al Nucleic Acids Res. 29 e115 (2001)). While the repression system is suitable for the expression of toxic genes and dominant negative mutants, the necessity to treat the parasites continuously and anhydrotetracycline (ATc) during the procedures of selection and cloning render it inappropriate for the generation of conditional knockouts.

There exists a need therefore to overcome this considerable limitation to the further study of Apicomplexan parasites to enable the application of techniques based on inducible transactivator technology.

### SUMMARY OF INVENTION

According to a first aspect of the present invention, there is provided a nucleic acid construct comprising the tetracycline repressor (TetR) operatively linked to a transacting factor of T. gondii.

According to a second aspect of the invention, there is provided a host cell transformed with a nucleic acid construct according to the first aspect. The host cell can be a bacterium, for example Escherichia coli, a yeast cell, for example Saccharomyces cerevisiae, or Schizosaccharomyces pombe, or a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia.

According to a third aspect of the invention, there is provided a nucleic acid construct according to the first aspect of the invention for use in medicine. This aspect of the invention therefore extends to a method of treatment for or prevention of an infection caused by a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia comprising administration of a nucleic acid construct according to the first aspect of the invention.

According to a fourth aspect of the invention, there is provided a vaccine composition comprising a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia transfected with a nucleic acid construct according to the first aspect of the invention.

According to a fifth aspect of the invention, there is provided the use of a nucleic acid construct according to the first aspect of the invention in the preparation of a vaccine for use in the treatment or prophylaxis of an infection caused by protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia.

According to a sixth aspect of the invention, there is provided a process for the preparation of a nucleic acid construct according to the first aspect of the invention, the process comprising ligating together nucleic acid sequences encoding a tetracycline-controlled transactivator and a transacting factor of T. gondii, optionally including linker or additional sequences.

According to a seventh aspect of the invention, there is provided a process for the preparation of a host cell according to the second aspect of the invention, the process comprising transfecting a cell with a nucleic acid construct according to the first aspect of the invention.

According to a eighth aspect of the invention, there is provided a process for the preparation of a vaccine composition according to the fourth aspect of the invention, the process comprising transfecting a host cell with a nucleic acid construct according to the first aspect of the invention.

Other aspects and advantages of the invention will be apparent from the following description and the appended claims.

### BRIEF DESCRIPTION OF DRAWINGS

FIGS. 1(a)-1(d) show trapping of a functional transactivator in T. gondii. FIG. 1(a) shows the scheme of the transactivator-TRAP strategy. FIG. 1(b) shows the clone Tati-1 regulated LacZ expression in ATc-dependent manner. FIG. 1(c) shows the amino acid sequence of the transactivating domain of TATi-1 (lower sequence line, starting “-PTF”) fused to tetR (upper sequence line, starting “..HQ”). FIG. 1(d) shows transient transfection of p7TetOS1LacZ-CAT into RH parasites or in strains expressing TATi or tTA^{2s}.

FIGS. 2(a)-2(e) show the generation of a conditional knockout for TgMyoA gene. FIG. 2(a) shows modulation of mycMyoA transgene expression in TATi-1 parasites. FIG. 2(b) shows Western blot analysis of mycMyoA expression. FIG. 2(c) shows detection of endogenous MyoA and myMyoA genes by analytical PCR on genomic DNA from RH, TATi-1 transformed with T7S4mycMyoA, myoako1 and myoako2. FIG. 2(d) shows analysis of myoako1 by Western blot with anti-MyoA antibodies which reveals that this clone lacked endogenous TgMyoA. FIG. 2(e) shows inducibility of mycMyoA expression in clones myoako1 and myoako2 which have regulatable mycMyoA.

FIGS. 3(a)-3(d) show phenotypic consequences of TgMyoA depletion for parasitic propagation in culture. FIG. 3(a) shows plaque assay for myoako1 and RH-wt. FIG. 3(b) shows invasion-assay of myoako1 in comparison to RH-wt parasites. FIG. 3(c) shows egression assay of myoako. FIG. 3(d) shows quantification of egress in function of incubation times after addition of Ca-ionophore A23187.

FIGS. 4(a) and 4(b) show the parasites depleted in TgMyoA are avirulent in mice and confer protection against new challenge with wild-type parasites. FIG. 4(a) shows tachyzoites from RH or myoako1 mutants that were injected i.p. into BALB/c mice and monitored for more than 30 days. FIG. 4(b) shows the development/induction of T. gondii-specific T cells after infection with myoako1 mutants.

FIGS. 5(a)-5(j) show the plasmid maps (with the respective nucleotide sequences shown in the sequence listing) for the vectors referred to in the Examples and FIGS. 1 to 4. FIGS. 5(a) to (d) show the vectors used to conditionally express MyoA or GFP: FIG. 5(a) shows pTetO7Sag4-MyoA, FIG. 5(b) shows pTetO7Sag1-MyoA, FIG. 5(c) shows pTetO7Sag4-GFP, and FIG. 5(d) shows pTetO7Sag1-GFP. FIGS. 5(e) to (g) show the vectors used to generate the recipient strain for the transactivator screening by random insertion: FIG. 5(e) shows pTetO7Sag1-HXGPRT, FIG. 5(f) shows pTetO7Sag1-LacZ, and FIG. 5(g) shows pTetO7Sag4-LacZ. FIG. 5(h) shows the vector used for the random integration pTub8TetRsynthetic: Ptub8TetR-GCN5-DHFRTS. FIGS. 5(i) and 5(j) show the vectors used to express a functional transactivator: FIG. 5(i) shows pTub8TATi-1-HXGPRT, and FIG. 5(j) shows pTub8TATi-3-HXGPRT.

FIGS. 6(a) and 6(b) show the nucleic acid sequences and presumed amino acid sequences of TATi-1 and TATi-3, respectively.

### DETAILED DESCRIPTION

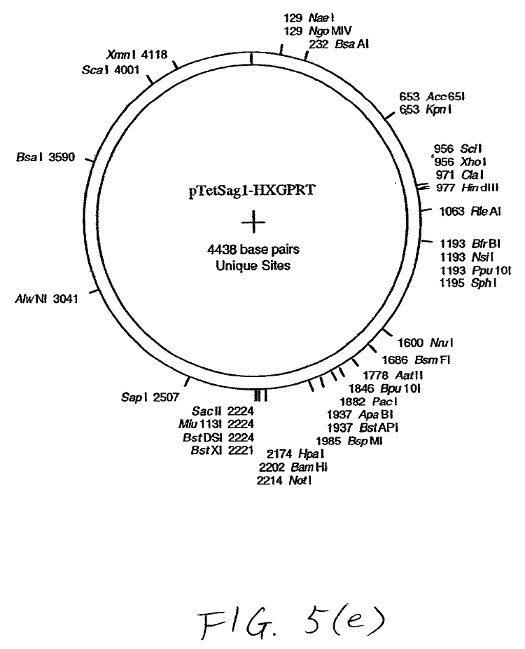

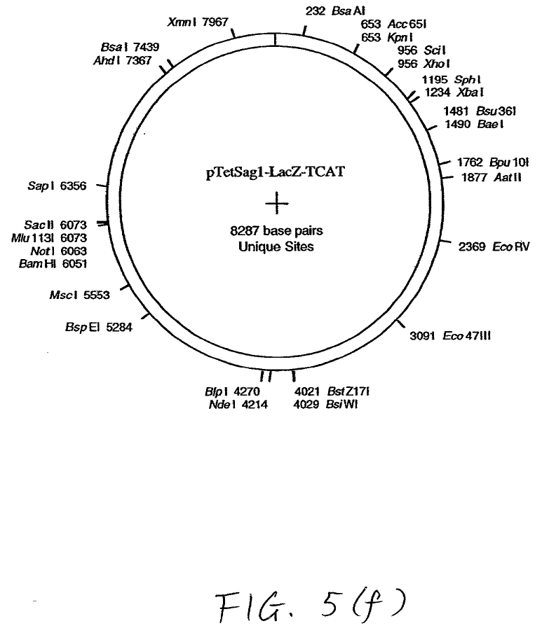

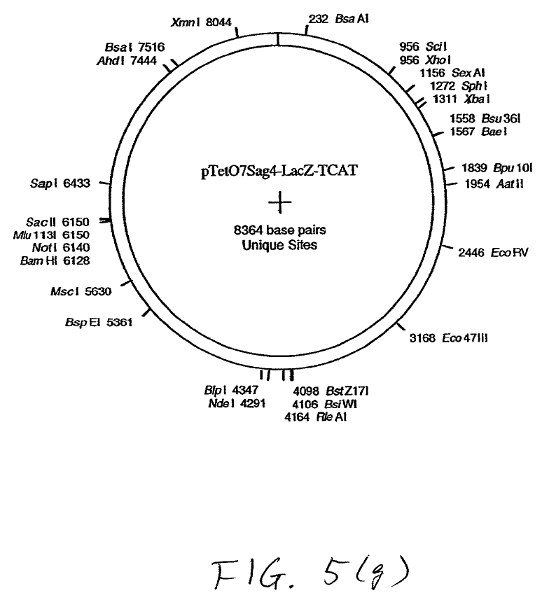

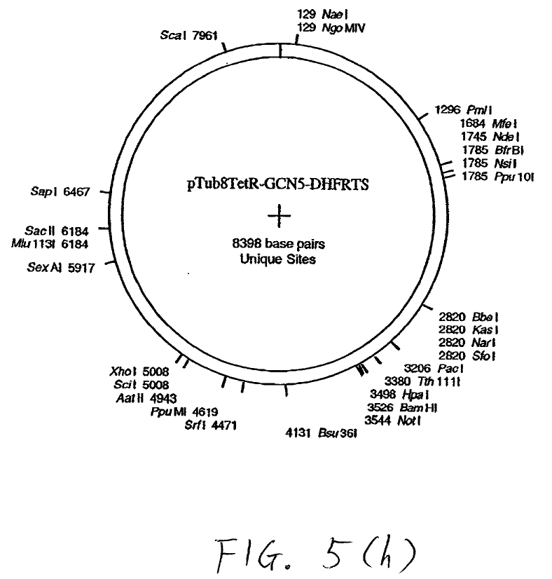

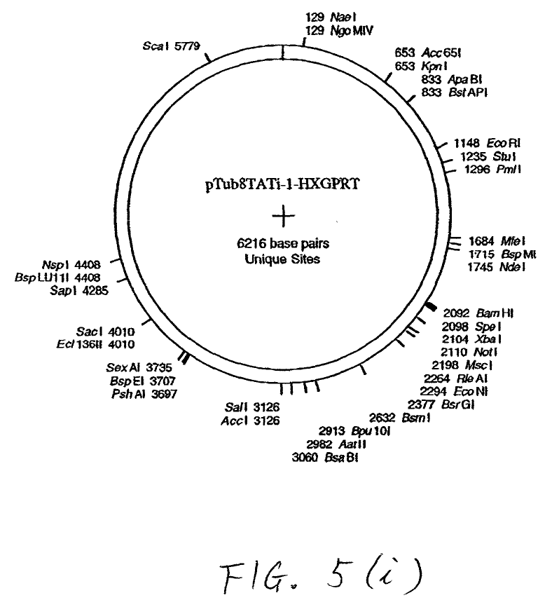

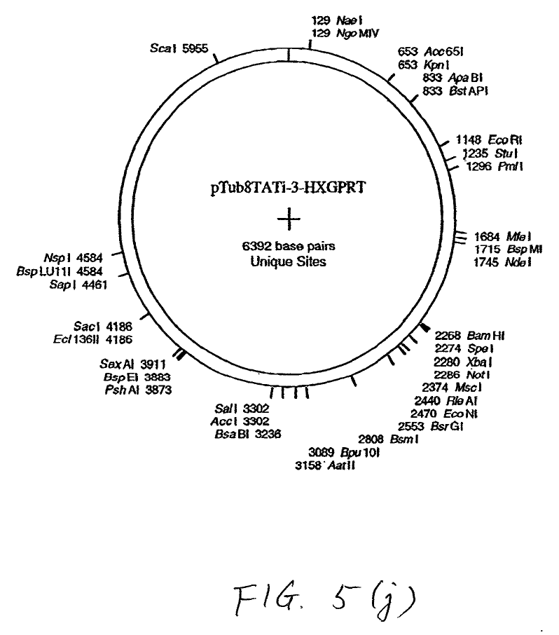

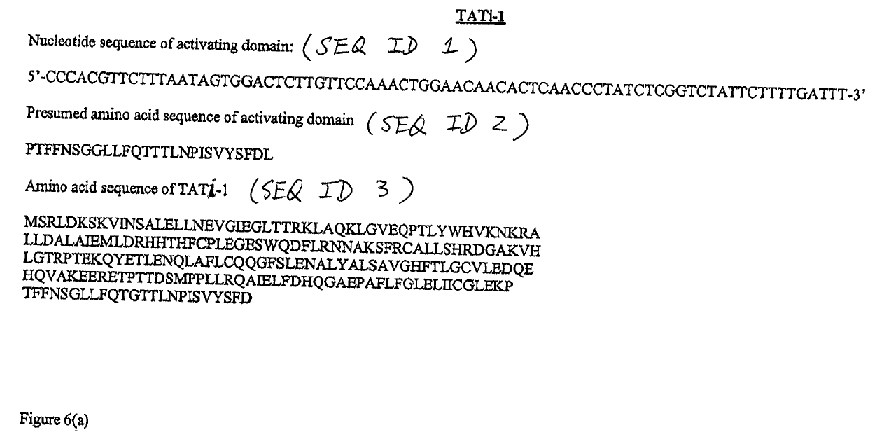

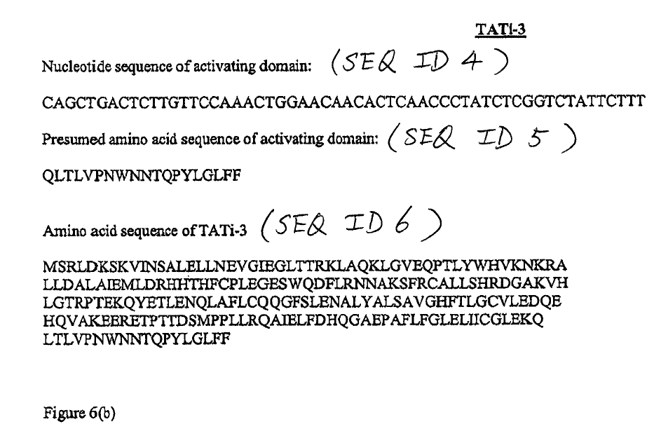

The TetR is described in Gossen, M. & Bujard, H. (1992)(Proc. Nat'l Acad. Sci USA 89 5547-5551) and sequence elements are shown in the constructs of FIG. 5. Transacting factors of T. gondii can be TATi-1 or TATi-3 in which the factor comprises a fusion protein of TetR and a T. gondii activating domain having a sequence as shown in FIG. 6. Alternatively, additional transacting factors can be identified using the methodology described in the present application. For example, using a library of degenerated oligonucleotides fused to the TetR could lead to the identification of artificial transcriptional activating domains.

The transacting factor may be TATi-1, TATi-3, or an analog, homolog, ortholog, related polypeptide, derivative, fragment or isoform thereof. The fusion protein formed between TetR and the activating domain may be a contiguous fusion of the two peptide sequences, or one or more additional linker amino acids may be inserted between the protein domains. Alternatively, one or more C-terminal residues from TetR may be truncated, N-terminal residues from the T. gondii activating domain.

The term “analog” as used herein refers to a polypeptide that possesses a similar or identical function as a transacting factor of T. gondii (TATi) but need not necessarily comprise an amino acid sequence that is similar or identical to the amino acid sequence of the TATi, or possess a structure that is similar or identical to that of the TATi. As used herein, an amino acid sequence of a polypeptide is “similar” to that of a TATi if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the TATi; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the SPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the TATi. As used herein, a polypeptide with “similar structure” to that of a TATi refers to a polypeptide that has a similar secondary, tertiary or quarternary structure as that of the TATi. The structure of a polypeptide can determined by methods known to those skilled in the art, including but not limited to, X-ray crystallography, nuclear magnetic resonance, and crystallographic electron microscopy.

Similarity of TATi polypeptides can also be determined functionally by transfecting a suitable host cell with a nucleic acid construct containing DNA encoding the polypeptide and monitoring for transactivating activity as herein described.

The term “TATi fusion protein” as used herein refers to a polypeptide that comprises (i) an amino acid sequence of a TATi, a TATi fragment, a TATi-related polypeptide or a fragment of an TATi-related polypeptide and (ii) an amino acid sequence of a heterologous polypeptide (i.e., a non-TATi, non-TATi fragment or non-TATi-related polypeptide), which will generally be TetR.

The term “TATi homolog” as used herein refers to a polypeptide that comprises an amino acid sequence similar to that of a TATi but does not necessarily possess a similar or identical function as the TATi.

The term “TATi ortholog” as used herein refers to a non-T. gondii polypeptide that (i) comprises an amino acid sequence similar to that of a TATi and (ii) possesses a similar or identical function to that of the TATi.

The term “TATi-related polypeptide” as used herein refers to a TATi homolog, a TATi analog, an isoform of TATi, a TATi ortholog, or any combination thereof.

The term “derivative” as used herein refers to a polypeptide that comprises an amino acid sequence of a second polypeptide which has been altered by the introduction of amino acid residue substitutions, deletions or additions. The derivative polypeptide possess a similar or identical function as the second polypeptide.

The term “fragment” as used herein refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of an SPI may or may not possess a functional activity of the second polypeptide.

The term “isoform” as used herein refers to variants of a polypeptide that are encoded by the same gene, but that differ in their pI or MW, or both. Such isoforms can differ in their amino acid composition (e.g. as a result of alternative splicing or limited proteolysis) and in addition, or in the alternative, may arise from differential post-translational modification (e.g., glycosylation, acylation, phosphorylation). As used herein, the term “isoform” also refers to a protein that exists in only a single form, i.e., it is not expressed as several variants.

The term “modulate” when used herein in reference to expression or activity of a TATi or a TATi-related polypeptide refers to the upregulation or downregulation of the expression or activity of the TATi or a TATi-related polypeptide. Based on the present disclosure, such modulation can be determined by assays known to those of skill in the art or described herein.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al, Nucleic Acids Res. (1997) 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

In the present invention, the transacting factor of T. gondii may be TATi, although it is envisaged that alternative synthetic forms of the polypeptide could be made by substitution of one or more amino acids in the molecule. The invention therefore extends to the use of a molecule having TATi activity. The skilled person is aware that various amino acids have similar properties. One or more such amino acids of a substance can often be substituted by one or more other such amino acids without eliminating a desired activity of that substance. Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Amino acid deletions or insertions may also be made relative to the amino acid sequence of TATi. Thus, for example, amino acids which do not have a substantial effect on the activity of TATi, or at least which do not eliminate such activity, may be deleted. Amino acid insertions relative to the sequence of TATi can also be made. This may be done to alter the properties of a substance of the present invention (e.g. to assist in identification, purification or expression, where the protein is obtained from a recombinant source, including a fusion protein. Such amino acid changes relative to the sequence of TATi from a recombinant source can be made using any suitable technique e.g. by using site-directed mutagenesis. The TATi molecule may, of course, be prepared by standard chemical synthetic techniques, e.g. solid phase peptide synthesis, or by preparation of nucleic acid encoding TATi, and subsequently expression of the nucleic acid in a suitable host cell system.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

Whatever amino acid changes are made (whether by means of substitution, insertion or deletion), preferred polypeptides of the present invention have at least 50% sequence identity with a polypeptide as defined in a) above more preferably the degree of sequence identity is at least 75%. Sequence identities of at least 90% or at least 95% are most preferred.

The degree of amino acid sequence identity can be calculated using a program such as “bestfit” (Smith and Waterman, Advances in Applied Mathematics, 482-489 (1981)) to find the best segment of similarity between any two sequences. The alignment is based on maximising the score achieved using a matrix of amino acid similarities, such as that described by Schwarz and Dayhof (1979) Atlas of Protein Sequence and Structure, Dayhof, M. O., Ed pp 353-358. Where high degrees of sequence identity are present there will be relatively few differences in amino acid sequence.

The nucleic acid encoding the transacting sequence of T. gondii can be a sequence complementary to, or homologous with the nucleic sequence for TATi-1 or TATi-3.

A nucleic acid sequence which is complementary to a nucleic acid sequence useful in a method of the present invention is a sequence which hybridises to such a sequence under stringent conditions, or a nucleic acid sequence which is homologous to or would hybridise under stringent conditions to such a sequence but for the degeneracy of the genetic code, or an oligonucleotide sequence specific for any such sequence. The nucleic acid sequences include oligonucleotides composed of nucleotides and also those composed of peptide nucleic acids. Where the nucleic sequence is based on a fragment of the gene encoding TATi, the fragment may be at least any ten consecutive nucleotides from the gene, or for example an oligonucleotide composed of from 20, 30, 40, or 50 nucleotides.

Stringent conditions of hybridisation may be characterised by low salt concentrations or high temperature conditions. For example, highly stringent conditions can be defined as being hybridisation to DNA bound to a solid support in 0.5M NaHPO₄, 7% sodium dodecyl sulfate (SDS), 1 mM EDTA at 65° C. and washing in 0.1×SSC/0.1% SDS at 68° C. (Ausubel et al eds. “Current Protocols in Molecular Biology” 1, page 2.10.3, published by Green Publishing Associates, Inc. and John Wiley & Sons, Inc., New York, (1989)). In some circumstances less stringent conditions may be required. As used in the present application, moderately stringent conditions can be defined as comprising washing in 0.2×SSC/0.1% SDS at 42° C. (Ausubel et al (1989) supra). Hybridisation can also be made more stringent by the addition of increasing amounts of formamide to destabilise the hybrid nucleic acid duplex. Thus particular hybridisation conditions can readily be manipulated, and will generally be selected according to the desired results. In general, convenient hybridisation temperatures in the presence of 50% formamide are 42° C. for a probe which is 95 to 100% homologous to the target DNA, 37° C. for 90 to 95% homology, and 32° C. for 70 to 90% homology.

Examples of preferred nucleic acid sequences for use in a method of the present invention are shown in the attached Figures.

The nucleic acid constructs of this aspect of the invention can be provided in the form of vectors, suitably expression vectors.

The term “vector” or “expression vector” generally refers to any nucleic acid vector which may be RNA, DNA or cDNA.

The term “expression vector” may include, among others, chromosomal, episomal, and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propogate or express nucleic acid to express a polypeptide in a host may be used for expression in this regard.

In certain embodiments of the invention, the vectors may provide for specific expression. Such specific expression may be inducible expression or expression only in certain types of cells or both inducible and cell-specific. Preferred among inducible vectors are vectors that can be induced for expression by environmental factors that are easy to manipulate, such as temperature and nutrient additives. Particularly preferred among inducible vectors are vectors that can be induced for expression by changes in the levels of chemicals, for example, chemical additives such as antibiotics. A variety of vectors suitable for use in the invention, including constitutive and inducible expression vectors for use in prokaryotic and eukaryotic hosts, are well known and employed routinely by those skilled in the art.

Recombinant expression vectors will include, for example, origins of replication, a promoter preferably derived from a highly expressed gene to direct transcription of a structural sequence, and a selectable marker to permit isolation of vector containing cells after exposure to the vector.

Expression vectors may comprise an origin of replication, a suitable promoter and enhancer, and also any necessary ribosome binding sites, polyadenylation regions, splice donor and acceptor sites, transcriptional termination sequences, and 5′-flanking non-transcribed sequences that are necessary for expression. Preferred expression vectors according to the present invention may be devoid of enhancer elements.

The promoter sequence may be any suitable known promoter, for example the human cytomegalovirus (CMV) promoter, the CMV immediate early promoter, the HSV thymidine kinase promoter, the early and late SV40 promoters or the promoters of retroviral LTR's, such as those of the Rous sarcoma virus (“RSV”), and metallothionein promoters, such as the mouse metallothionein-I promoter. The promoter may comprise the minimum sequence required for promoter activity (such as a TATA box without enhancer elements), for example, the minimal sequence of the CMV promoter (mCMV). The promoter, if present, can be contiguous to the TetR sequence.

The expression vectors or vectors of the invention can be derived from a vector devoid of its own promoter and enhancer elements, for example the plasmid vector PGL2. Enhancers are able to bind to promoter regions situated several thousands of bases away through DNA folding (Rippe et al TIBS 1995; 20: 500-506 (1995)).

The expression vectors may also include selectable markers, such as antibiotic resistance, which enable the vectors to be propagated.

The nucleic acid sequence of the first aspect of the invention may additionally comprise a reporter transcription unit lacking a promoter region, such as a chloramphenicol acetyl transferase (“CAT”) or DHFR-TS transcription unit. As is well known, introduction into an expression vector of a promoter-containing fragment at a restriction site upstream of the CAT gene engenders the production of CAT activity, which can be detected by standard CAT assays. The application of reporter genes relates to the phenotype of these genes which can be assayed in a transformed organism and which is used, for example, to analyse the induction and/or repression of gene expression. Reporter genes for use in studies of gene regulation include other well known reporter genes including the lux gene encoding luciferase which can be assayed by a bioluminescence assay, the uidA gene encoding β-glucuronidase which can be assayed by a histochemical test, the aphIV gene encoding hygromycin phosphotransferase which can be assayed by testing for hygromycin resistance in the transformed organism, the dhfr gene encoding dihydrofolate reductase which can be assayed by testing for methotrexate resistance in the transformed organism, the neo gene encoding neomycin phosphotransferase which can be assayed by testing for kanamycin resistance in the transformed organism and the lacZ gene encoding β-galactosidase which can be assayed by a histochemical test. All of these reporter genes are obtainable from E. coli except for the lux gene. Sources of the lux gene include the luminescent bacteria Vibrio harveyii and V. fischeri, the firefly Photinus pyralis and the marine organism Renilla reniformis.

The invention can also be described as providing an Apicomplexan tetracycline-inducible transactivator (TATi) system, comprising the tetracycline repressor (TetR) and a transacting factor of T. gondii. Alternatively, it can be described as providing a tetracycline-inducible transactivator (TATi) system, comprising the tetracycline repressor (TetR) and a transacting factor of T. gondii for use in Apicomplexan species.

The advantages of the invention extend to the production of live attenuated vaccines suitable to prevent infection by Apicomplexan parasites, the provision of a system to permit the generation of conditional knock-outs of essential gene(s) in such parasites leading to a greater understanding of the parasites metabolism which may allow for the design of new pharmaceutical agents to block or inhibit the function of the essential gene(s). This system allows for the identification of essential genes and for the validation of such genes as drug targets or vaccine candidates.

To unravel the function of essential genes in an Apicomplexan parasite, for example Toxoplasma gondii or Plasmodium species, the present invention has established a tetracycline-inducible transactivator system (TATi), which ectopically controls gene expression. In a mutant T. gondii strain expressing TATi, a second copy of a gene of interest can be introduced into the cell under the control of the Tet promoter, and the function of the native gene disrupted, for example by homologous recombination, or another gene targeted insertion sufficient to prevent normal gene function. The mutant obtained by this procedure is a fully conditional mutant thus enabling study of the gene concerned.

According to a second aspect of the invention, there is provided a host cell transformed with a nucleic acid construct according to the first aspect. The host cell can be a bacterium, for example Escherichia coli, a yeast cell, for example Saccharomyces cerevisiae, or Schizosaccharomyces pombe, or a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia.

Introduction of an expression vector into the host cell can be effected by calcium phosphate transfection, DEAE-dextran mediated transfection, microinjection, cationic lipid-mediated transfection, electroporation, transduction, scrape loading, ballistic introduction, infection of other methods. Such methods are described in many standard laboratory manuals, such as Sambrook et al., Molecular Cloning: A Laboratory Manual, 2^{nd} Ed., Cold Spring Harbor Laboratory Press, Cold Spring Harbor, N.Y. (1989).

According to a third aspect of the invention, there is provided a nucleic acid construct according to the first aspect of the invention for use in medicine. Typically, such uses will be for the treatment or prevention of infections caused by a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia. This aspect of the invention therefore extends to a method of treatment for or prevention of an infection caused by a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambiacomprising administration of a nucleic acid construct according to the first aspect of the invention.

Malaria is a disease condition caused in animals by the Plasmodium spp parasites characterised by fever in mild forms and by metabolic acidosis, severe anaemia and cerebral malaria, in severer forms, and sometimes in the death of the subject infected. It is also possible for asymptomatic infection to occur in some affected subjects. In humans, the disease is caused by Plasmodium falciparum and to a lesser extent by Plasmodium vivax where the parasites are transmitted by Anopheles spp mosquitoes. Malaria is caused in rodents by Plasmodium berghei, Plasmodium yoelii and in rhesus monkeys by Plasmodium knowlesi.

According to a fourth aspect of the invention, there is provided a vaccine composition comprising a protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia transfected with a nucleic acid construct according to the first aspect of the invention.

According to a fifth aspect of the invention, there is provided the use of a nucleic acid construct according to the first aspect of the invention in the preparation of a vaccine for use in the treatment or prophylaxis of an infection caused by protozoan, for example Toxoplasma gondii or Plasmodium species, for example Plasmodium falciparum, Plasmodium vivax, Plasmodium berghei, Plasmodium yoelii or Plasmodium knowlesi, or Trypanosoma brucei, or Entamoeba histolytica, or Giardia lambia.

According to a sixth aspect of the invention, there is provided a process for the preparation of a nucleic acid construct according to the first aspect of the invention, the process comprising ligating together nucleic acid sequences encoding a tetracycline-controlled transactivator and a transacting factor of T. gondii, optionally including linker or additional sequences.

According to a seventh aspect of the invention, there is provided a process for the preparation of a host cell according to the second aspect of the invention, the process comprising transfecting a cell with a nucleic acid construct according to the first aspect of the invention.

According to a eighth aspect of the invention, there is provided a process for the preparation of a vaccine composition according to the fourth aspect of the invention, the process comprising transfecting a host cell with a nucleic acid construct according to the first aspect of the invention.

As described above, the nucleic acid constructs, vectors or expression vectors of the invention can be used in medicine, and the invention therefore extends to compositions comprising the nucleic acid construct according to the first aspect and embodiments of the subsequent aspects as appropriate of the invention. Therefore, the nucleic acid constructs, vectors, or expression vectors or systems of the present invention may be employed in combination with a pharmaceutically acceptable carrier or carriers.

Such carriers may include, but are not limited to, saline, buffered saline, dextrose, liposomes, water, glycerol, ethanol and combinations thereof.

The nucleic acid construct, expression vector or vectors of the invention may be employed alone or in conjunction with other compounds, such as therapeutic compounds.

The pharmaceutical compositions may be administered in any effective, convenient manner effective for treating a patients disease including, for instance, administration by oral, topical, intravenous, intramuscular, intranasal, or intradermal routes among others. In therapy or as a prophylactic, the active agent may be administered to an individual as an injectable composition, for example as a sterile aqueous dispersion, preferably isotonic.

The invention also provides a kit of parts comprising a nucleic acid construct, expression vector or vector of the invention as defined above and an administration vehicle including, but not limited to, tablets for oral administration, inhalers for lung administration and injectable solutions for intravenous administration.

Preferred features of the second and subsequent features of the invention are as for the first aspect mutatis mutandis.

The invention will now be further described by way of example with reference to the following Examples which are present for the purposes of illustration only and are not to be construed as being limiting on the invention. In the Examples reference is made to a number of Figures in which:

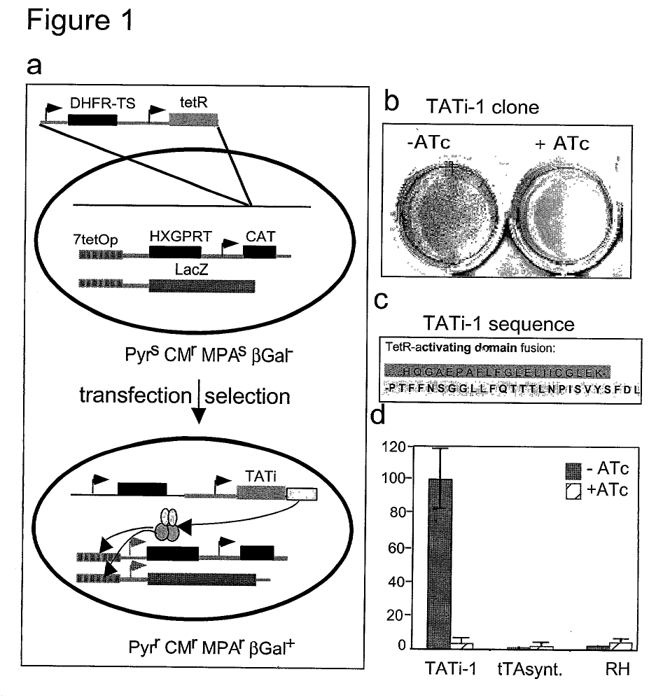

FIG. 1 shows trapping of a functional transactivator in T. gondii. FIG. 1(a) shows the scheme of the transactivator-TRAP strategy. A linear DNA fragment encoding dhfrts selectable marker and the TetR without a STOP was stably integrated at random into the genome of the recipient strain. Upon integration in a locus where a functional transactivator fusion has been generated, the TetR-fusion activates expression of the selectable marker gene HXGPRT and of reporter gene LacZ. FIG. 1(b) shows the clone Tati-1 regulated LacZ expression in ATc-dependent manner. Parasites were grown for 48 hours in the presence of and absence of drug, fixed and stained with X-Gal to monitor LacZ expression as performed previously (Meissner et al Nucleic Acids Res. 29 e115 (2001)). FIG. 1(c) shows the amino acid sequence of the transactivating domain of TATi-1 (lower sequence line, starting “-PTF”) fused to tetR (upper sequence line, starting “..HQ”). FIG. 1(d) shows transient transfection of p7TetOS1LacZ-CAT into RH parasites or in strains expressing TATi or tTA^{2s}. Cells were grown for 48 hours in the presence or absence of ATc before parasite lysates were prepared to quantify LacZ expression.

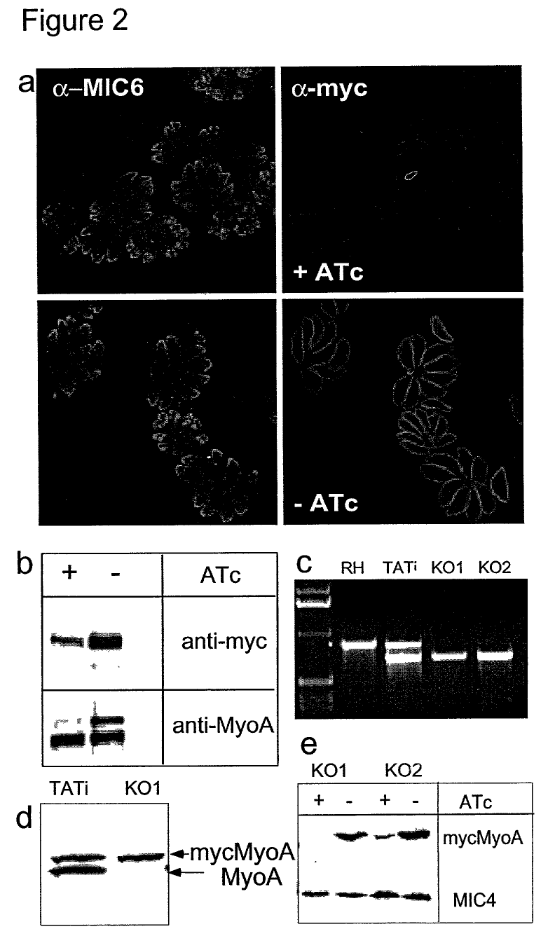

FIG. 2 shows the generation of a conditional knockout for TgMyoA gene. FIG. 2(a) shows modulation of mycMyoA transgene expression in TATi-1 parasites. Detection of mycMyoA by IFA, on intracellular parasites incubated in the presence of or absence of ATc for 48 hours, using mAb anti-myc. MIC4 was detected under the same setting and exposure time with rabbit polyclonals as control. FIG. 2(b) shows Western blot analysis of mycMyoA expression. Parasite lysates were probed with monoclonal anti-myc and polyclonal anti-MyoA. As internal standard, the lysate was probed with polyclonal anti-MIC4. The upper band corresponds to the inducible mycMyoA. Parasites were grown for 48 hours in the presence of or absence of ATc before lysates were prepared. FIG. 2(c) shows detection of endogenous MyoA and myMyoA genes by analytical PCR on genomic DNA from RH, TATi-1 transformed with T7S4mycMyoA, myoako1 and myoako2. Sequence specific primers were used to amplify endogenous TgMyoA (in an intron) and transgenic mycMyoA (in the 5′-UTR). FIG. 2(d) shows analysis of myoako1 by Western blot with anti-MyoA antibodies which reveals that this clone lacked endogenous TgMyoA. FIG. 2(e) shows inducibility of mycMyoA expression in clones myoako1 and myoako2 which have regulatable mycMyoA. Intracellular parasites were grown for 48 hours in the presence of or absence of ATc before preparation of total cell lysate. The level of detection of TgMIC4 was used as control for equal loading.

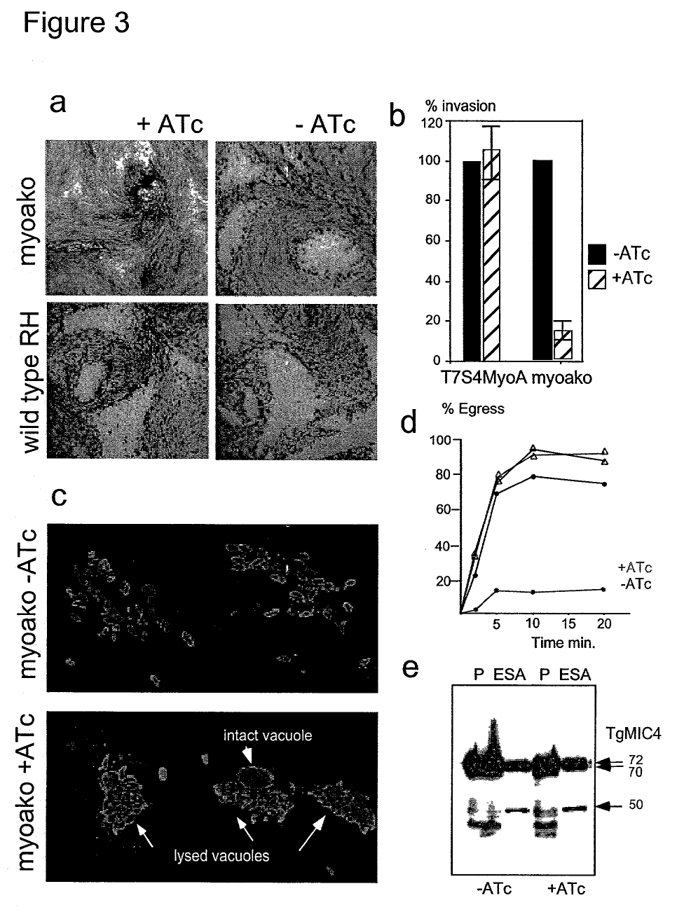

FIG. 3 shows phenotypic consequences of TgMyoA depletion for parasitic propagation in culture. FIG. 3(a) shows plaque assay for myoako1 and RH-wt. Parasites were continuously grown on HFF-monolayer in the presence of or absence of ATc for 10 days before fixation and staining of the cells with GIEMSA. FIG. 3(b) shows invasion-assay of myoako1 in comparison to RH-wt parasites. 5.10⁵ freshly lysed parasites grown in the presence or absence of ATc for 48 hours were inoculated on HFF-monolayer for 20 minutes followed by a washing step to remove extracellular parasites. Cells were further incubated for 24 hours before fixation. The number of vacuoles represented successful invasion events and were counted in 40 eye-fields for each parasite. 100% represents the number of vacuoles in absence of ATc for RH and myoako1 respectively. FIG. 3(c) shows egression assay of myoako. Parasites were grown for 36 hours of HFF-cells in presence of and absence of ATc. Cells were fixed 5 minutes after addition of Ca-ionophore A23187 according to Black et al (Mol. Cell. Biol. 20 9399-9408 (2000)).and analysed by IFA using anti-SAG1 antibodies. FIG. 3(d) shows quantification of egress in function of incubation times after addition of Ca-ionophore A23187.

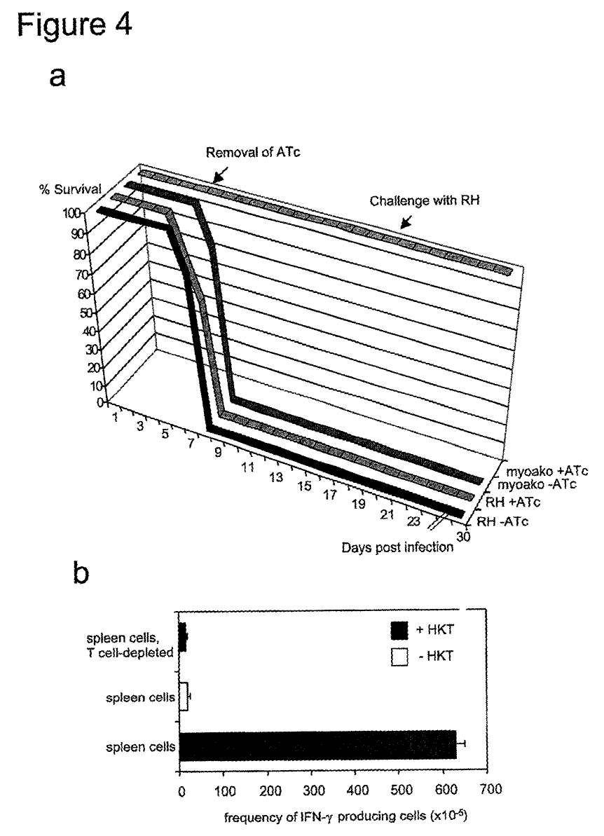

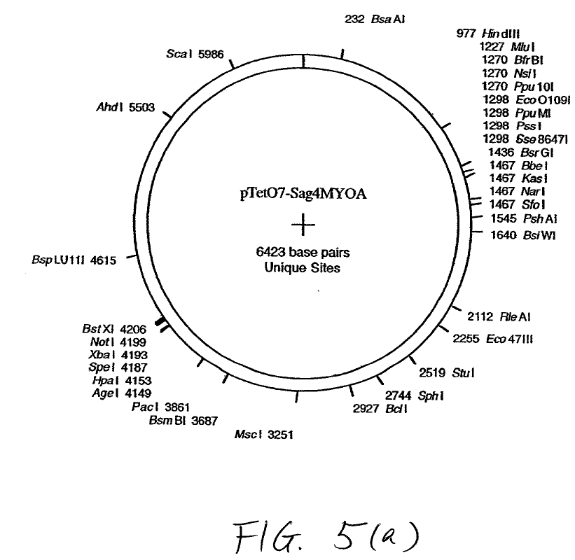

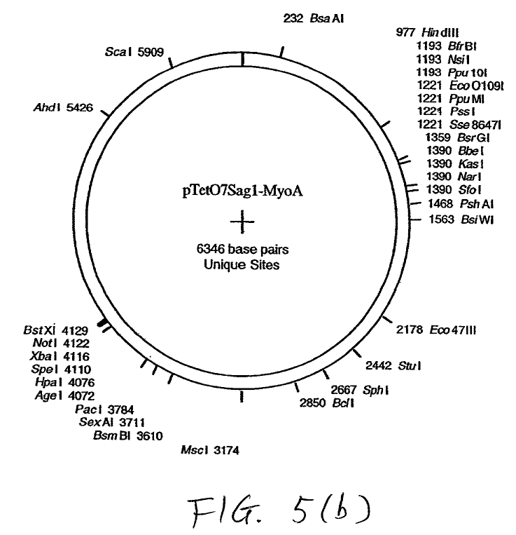

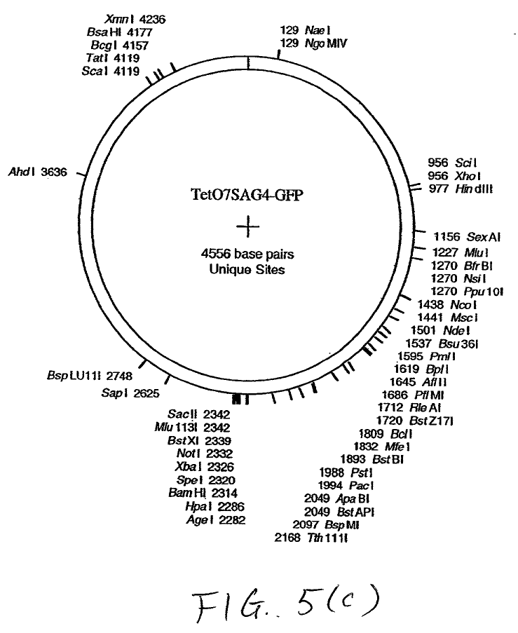

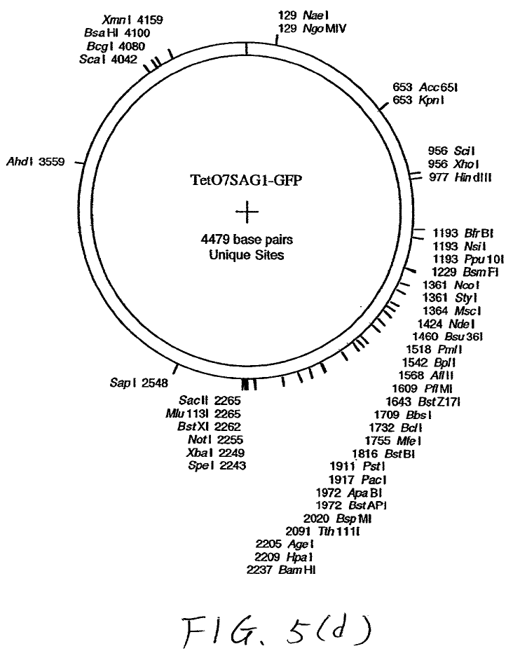

FIG. 4 shows the parasites depleted in TgMyoA are avirulent in mice and confer protection against new challenge with wild-type parasites. FIG. 4(a) shows tachyzoites from RH or myoako1 mutants that were injected i.p. into BALB/c mice and monitored for more than 30 days. Groups of mice were given 0.2 mg/ml ATc in drinking water or normal water. After 11 days, the group of 10 mice infected with myoako and treated with ATc survived and the drug was removed. After 17 days, 5 mice were challenged with RH wild-type parasites and survived the infection. FIG. 4(b) shows the development/induction of T. gondii-specific T cells after infection with myoako1 mutants. At day 21 after infection, spleens were isolated and the development of T. gondii-specific T cells was determined by IFN-γ-ELISPOT. The mean of triplicates±is shown.

FIG. 5 shows the plasmid maps and respective nucleotide sequences for the vectors referred to in the Examples and FIGS. 1 to 4.

FIGS. 5(a) to (d) show the vectors used to conditionally express MyoA or GFP: FIG. 5(a) shows pTetO7Sag4-MyoA, FIG. 5(b) shows pTetO7Sag1-MyoA, FIG. 5(c) shows pTetO7Sag4-GFP, and FIG. 5(d) shows pTetO7Sag1-GFP.

FIGS. 5(e) to (g) show the vectors used to generate the recipient strain for the transactivator screening by random insertion: FIG. 5(e) shows pTetO7Sag1-HXGPRT, FIG. 5(f) shows pTetO7Sag1-LacZ, and FIG. 5(g) shows pTetO7Sag4-LacZ.

FIG. 5(h) shows the vector used for the random integration pTub8TetRsynthetic: Ptub8TetR-GCN5-DHFRTS. FIGS. 5(i) and 5(j) show the vectors used to express a functional transactivator: FIG. 5(i) shows pTub8TATi-1-HXGPRT, and FIG. 5(j) shows pTub8TATi-3-HXGPRT.

FIG. 6 shows the nucleic acid sequences and presumed amino acid sequences of TATi-1 and TATi-3.

### EXAMPLE 1

Preparation of Tet-Inducible Transactivator Construct

T. gondii tachyzoites (RH hxgprf) were grown in human foreskin fibroblasts (HFF) and maintained in Dulbecco's modified Eagle's medium (DMEM) supplemented with 10% fetal calf serum (FCS), 2 mM glutamine and 25 μg/ml gentamicin. To generate stable transformants, 5.10⁷ freshly released RHhxgprf parasites were transfected and selected in presence of mycophenolic acid and xanthine (MPA/X) as previously described (Donald et al J. Biol. Chem. 271 14010-14019 (1996)). The selection based on chloramphenicol or pyrinmethamine resistance was achieved as described earlier (Soldat, D., & Boothroyd, J. C., Science 260 349-352 (1993); Donald, R. G. K., & Roos, D. S., Proc. Nat'l Acad. Sci. USA 90 11703-11707 (1993)). Homologous recombination was obtained as described previously (Reiss et al J. Cell. Biol. (2001)).

Plasmids

The reporter plasmids for the tet-Transactivator system, p7tetOHXGPRT, p7tetOLacZCAT were described previously (Meissner et al Nucleic Acids. Res. 29 e115 (2001)). For the construction of p7tetOS4mycMyoA the promoter region of p5RT70Tet4mycMyoA (Meissner et al Nucleic Acids. Res. 29 e115 (2001)) was exchanged by p7tetOS4 using NsiI/PacI. The resulting plasmid p7tetOS4mycMyoA was stably introduced by contransfection with pDHFRTs using DHFR-selection.

The construct pTgMyoA-kkoTCAT was composed of pT230 CAT previously described (Soldati, D. & Boothroyd, J. C., Mol. Cell. Biol. 15 87-93 (1995)) and flanked on both sides with 2.0 and 2.2 Kbp and of 5′- and 3′-flanking sequences of TgMyoA gene. The vector was linearised at both ends of the flanking sequences to rise the frequency of double homologous recombination.

The plasmid pTRep-DHFRTs was constructed in two steps. The TetR fragment was amplified with the oligonucleotides Rep-4 and Rep-7, which are:

Rep-4

5′CGGAATTCCTTTTCGACAAAATGTCGCGCCTGGACAAGAGCAAAGTCATCAACTCTGC-3′

Rep-7

5′CCCTTAATTAATGCATACCGCTTTCGCACTTCAGCTG-3′

The resulting PCR-fragment encoding TetR without a STOP-codon was cloned in the EcoRI/PacI sites of p5RT70GFP. In the second step, the TgDHFRTS selectable marker gene was inserted into the SacII-site.

The plasmid pTTATi-1-HX was constructed using the rescued RT-PCR fragment that was amplified using a poly-T-Primer with a BamH1-restriction site at the 3′-end and the tetR-specific primer Rep-4. The fragment was digested using EcoRI/BamHI and inserted between the same sites of p5RT70/HX. Recipient strain for the transactivator trapping was established by con-transformation of RHhxgprt⁻ with p7TetOS1LacZ-CAT and p7TetOHXGPRT. Stable parasites were selected using CAT. Integration of both plasmids was verified by analytical PCR.

Random insertion was used to identify a transactivating domain, which functions as tet-regulatable transactivator due to its fusion with the tetR. For this purpose, a construct expressing the tetR but lacking a stop codon was randomly integrated into T. gondii genome using the dehydrofolate reductase-thymidylate synthase (DHFR-TS) as selectable marker, which was previously shown to exhibit a very high frequency of integration (10⁻²). The recipient parasitic cell line used for the screening was deficient in HXGPRT gene and was transformed with two vectors expressing LacZ and HXGPRT under the control of a minimal promoter containing 7 tet-operator sequences (7tetO) (FIG. 1a). One of the parasite mutants resistant to mycophenolic acid and expressing LacZ in a tet-dependent fashion was characterised further. The tetR fusion was rescued by RT-PCR, cloned and sequenced. The fusion consisting of a 26 amino acids attached in the C-term of tetR was named TATi-1 (FIG. 1b). A new expression vector for TATi-1, pTTATi-1-HX, was constructed shown to confer tet-dependent LacZ expression when transiently transfected into a parasitic strain containing p7TetOLacZ. A stable line expressing TATi-1 using HXGPRT as selection was generated in RH to establish a recipient strain for the tet-system.

### EXAMPLE 2

Plaque, Invasion and Egression Assays

Among many vital functions in obligate intracellular parasites, the process of host cell invasion is prerequisite for their survival and replication. Penetration into host cells is an active process dependent on parasite motion. Gliding motility has been shown previously demonstrated to require an intact actin cytoskeleton and to be powered by a myosin motor. The small unconventional myosin A (TgMyoA) is the primary candidate which localises beneath the plasma membrane (Heintzelman, M. B. & Schwartzman, J. D. J. Mol. Biol. 271 139-146 (1997); Hettmann et al Mol. Cell. Biol. 11 1385-1400 (2000)) and exhibits all the biochemical and biophysical properties necessary to generate fast movements. Consistent with an essential role for parasite survival, all our attempts to disrupt TgMyoA gene failed so far.

A second copy of TgMyoA under the control of the tet-promoter was introduced into the TATi-1 expressing cell line and monitored the modulation of TgMyoA transgene expression upon treatment of ATc by Western blot and indirect immunofluorescence (FIG. 2a, b). Using this robust inducible tet-system, we were able to disrupt the endogenous TgMyoA gene by homologous recombination with a vector carrying 5′- and 3′-TgMyoA flanking sequences respectively and chloramphenicol acetyltransferase as selectable marker. The absence of endogenous copy was determined by genomic PCR and by Western blot (FIG. 2c). The anticipated role of TgMyoA in parasite motion implied that parasites lacking the protein would be impaired in host cell invasion, egression and spreading. The phenotypic consequences of TgMyoA depletion could be best visualised in a plaque assay (FIG. 3a). After inoculation of HFF monolayers, the mutant parasites were cultivated with ATc over a period of 5-7 days showed an inability to form plaques in the HFF monolayers while non-treated parasites formed large plaques of lysis. This process was reversible upon removal of ATc (data not shown). The process of host cell penetration was examined specifically by invasion assay using freshly lysed parasites previously cultivated in presence or absence of ATc for 72 hours. The invasion rate was determined 24 hours later by counting the number of vacuoles (FIG. 3b). The number of parasites per vacuoles was identical for control and conditional myoako parasites, treated or not with ATc, indicating that the depletion in TgMyoA did not affect intracellular growth. Parasites use the same machinery to penetrate and egress host cells, so we performed an egression assay using the calcium ionophore for a short period of 5 min as previously described, followed by fixation and visualisation by IFA using anti-surface antigen 1 antibodies (SAG1) (FIG. 3c). Egression assay was performed as described previously (Black et al Mol. Cell. Biol. 20 9399-9408 (2000)). The regulated exocytosis by the apical organelles called micronemes plays a critical role in gliding motility and invasion. The micronemes secrete complexes of transmembrane and soluble adhesins upon raise in parasite intracellular calcium. These complexes are interacting with host cell receptors and their redistribution toward the posterior pole is driving gliding motion. To exclude any involvement of TgMyoA in microneme exocytosis, that would explain the phenotypes observed, we examined the discharge by micronemes. The secretion assay revealed no alteration of exocytosis upon TgMyoA depletion. (FIG. 3d).

### EXAMPLE 3

Murine Virulence Assay

Freshly lysed tachyzoites from infected HFF monolayers were washed in PBS and counted under the microscope. Tachyzoites were injected intraperitoneally (i.p) in 0.2 ml into mice aged 6-8 weeks. The actual p.f.u, in the inoculum was determined by plaque assay. Mice on anydrotetracycline treatment were housed five per cage and were given 1 mg/ml solution instead of normal drinking water.

RH is a type I strain of T. gondii, which typically kills mice with a LD100 of a single infectious organism. The conditional myoako mutant was assessed for virulence in mice. Both wild type and mutant parasites were inoculated intraperitoneally in two groups of mice supplemented or not with ATc in the drinking water. After even days, all the mice infected with the control and the conditional myoako still expressing TgMyoA transgene were dead (FIG. 4a.) In contrast, we observed 100% survival in the group of mice infected with myoako but supplemented with ATc 11 days post-infection. All the mice were serologically positive for T. gondii as monitored by immunoblot (data not shown). At day 21-post infection, two mice were sacrificed and analysed for the presence of T. gondii-specific T-cells in the spleen, as determined by an IFN-γ-specific ELISPOT. The analysis was positive (FIG. 4b), indicating that the mice infected by myoako in the presence of ATc have been able to raise a detectable humoral and a cellular response against the parasites. To determine if these mice were protected against subsequent challenge of RH, they were innoculated i.p. with 150 parasites of RH strain at day 17 post infection. All the mice survived the challenge indicating that the myoako induced a protective immunity.

The tet-system described here has been selected in T. gondii and is not active in the Hela cells (data not shown). The activating domain is capable of interacting specifically with the transcription machinery of the parasite and might function as a transactivator in the closely related parasite Plasmodium falciparum.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A nucleic acid construct comprising a tetracycline repressor operatively linked to a transacting factor of T. gondii.

2. A nucleic acid construct as claimed in claim 1, wherein the transacting factor of T. gondii comprises a nucleic acid sequence selected from a group consisting of TATi-1 activating domain, TATi-3 activating domain, and a sequence complementary or homologous thereto.

3. A transcriptional activator of T. gondii comprising an amino acid sequence of TATi-1 or TATi-3, or an analog, homolog, ortholog, related polypeptide, derivative, fragment or isoform thereof.

4. A transacting factor of T. gondii comprising an amino acid sequence of TATi-1 or TATi-3 activating domain, or an analog, homolog, ortholog, related polypeptide, derivative, fragment or isoform thereof.

5. A vector comprising a nucleic acid construct as defined in claim 1.

6. An expression vector comprising a nucleic acid construct as defined in claim 1.

7. An Apicomplexan tetracycline-inducible transactivator system, comprising a tetracycline repressor and a transacting factor of T. gondii.

8. A tetracycline-inducible transactivator system, comprising a tetracycline repressor and a transacting factor of T. gondii for use in Apicomplexan species.

9. A host cell transformed with a nucleic acid construct as defined in claim 1, or a vector as claimed in claim 4.

10. A host cell as claimed in claim 9, which is an Apicomplexan host cell.

11. A host cell as claimed in claim 10, in which the Apicomplexan cell is selected from the group consisting of Toxoplasma gondii, Plasmodium falciparum, Plasmodium vivax, Plasmodium berghei, Plasmodium yoelii, Plasmodium knowlesi, Trypanosoma brucei, Entamoeba histolytica, and Giardia lambia.

12. A nucleic acid construct as defined in claim 1 for use in medicine.

13. A host cell as defined in claim 9 for use in medicine.

14. A method of treatment for or prevention of an infection caused by a protozoan, selected from the group consisting of Toxoplasma gondii, Plasmodium falciparum, Plasmodium vivax, Plasmodium berghei, Plasmodium yoelii, Plasmodium knowlesi, Trypanosoma brucei, Entamoeba histolytica and Giardia lambia, comprising administration to a subject of a nucleic acid construct as defined in claim 1.

15. A method of treatment for or prevention of an infection caused by a protozoan, selected from the group consisting of Toxoplasma gondii, Plasmodium falciparum, Plasmodium vivax, Plasmodium berghei, Plasmodium yoelii, Plasmodium knowlesi, Trypanosoma brucei, Entamoeba histolytica and Giardia lambia, comprising administration to a subject of a host cell as defined in claim 9.

16. A method of treatment as claimed in claim 14, in which the protozoan is Toxoplasma gondii.

17. A method of treatment as claimed in claim 14, in which the protozoan is a Plasmodium species.

18. A vaccine composition comprising a protozoan selected from the group consisting of Toxoplasma gondii, Plasmodium falciparum, Plasmodium vivax, Plasmodium berghei, Plasmodium yoelii, Plasmodium knowlesi, Trypanosoma brucei, Entamoeba histolytica and Giardia lambia transfected with a nucleic acid construct as defined in claim 1.

19. The use of a nucleic acid construct as defined in claim 1 in the preparation of a vaccine for use in the treatment or prophylaxis of an infection caused by a protozoan selected from the group consisting of Toxoplasma gondii, Plasmodium falciparum, Plasmodium vivax, Plasmodium berghei, Plasmodium yoelii, Plasmodium knowlesi, Trypanosoma brucei, Entamoeba histolytica and Giardia lambia.

20. A kit of parts comprising a host cell as defined in claim 9 and an administration vehicle selected from tablets for oral administration, inhalers for lung administration, and injectable solutions for intravenous administration.


SEQUENCE LISTING (31 sequences)

SEQ ID NO: 1 - protein, 47 aa, Artificial Sequence
  Description of Artificial Sequence TetR-activating domain fusion
HQGAEPAFLFGLELIICGLEKPTFFNSGLLFQTGTTLNPISVYSFDL

SEQ ID NO: 2 - DNA, 12 bp, Artificial Sequence
  Description of Artificial Sequence pTetO7Sag4-MyoA
ctgacgcgccct

SEQ ID NO: 3 - DNA, 12 bp, Artificial Sequence
  Description of Artificial Sequence pTetO7Sag4-MyoA
gaaaagtgccac

SEQ ID NO: 4 - DNA, 6423 bp, Artificial Sequence
  CDS (1270)..(3864)
  Description of Artificial Sequence pTetO7Sag4-MyoA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttacgccgctgagactaactagaaagaagtgtgcaacagttcatgagggacaaaaggaatgtgatgcggttcgcttgaagaaggaatgtttaagcacgtcaacaatacgccttggcgaatgttcatgactgttcatgtggttcatcggatcatttgaaaacatcgtgaggctggtacctggtcgcaaacgtcgtagtgtagtaccgacaataacgtcgtcgttcaaggggacgcagttctcggaagacgcgtcgcagcatactgcaactgctttcgtctgtcttcaaccatgcatgagcagaagctcatctccgaggaggacctgctgcatcatcatcatcatcatcatgatggtaccgagctcgcgagcaagaccacgtctgaggagctgaaaacggccacggcgctgaagaagaggtcgtccgatgtccacgcggtcgaccactccggcaatgtgtacaaaggatttcaaatctggacggacttggcgccgtcggtgaaggaggagccggacctgatgtttgccaagtgcatcgtgcaggcggggacagacaaggggaacttgacctgcgtccagatcgatccaccgggcttcgacgaaccgttcgaagtcccgcaggcgaatgcgtggaacgtaaacagcctgatcgaccccatgacgtacggagacatcggcatgttgcctcacacgaacattccttgcgtcctcgacttcctcaaggtgcgcttcatgaagaatcaaatctacacgactgcggacccgctcgtcgtcgccatcaatcccttccgcgacctcgggaacaccacgctcgactggattgttcgatacagagacactttcgacctctccaaactcgcgccccatgttttctacaccgcccgacgcgcgctcgacaacctccacgccgtcaacaagtcgcaaacgatcatcgtgtccggtgagtctggcgcgggcaagacggaggcgacgaagcagattatgaggtattttgcggcggcgaagacggggtcgatggatttgcggattcagaacgcgatcatggcggcgaatccagtgcttgaggcatttggaaatgcgaagacgattcgcaacaacaactcgtcgcgtttcggacgcttcatgcagctggatgtgggtcgcgaaggaggcatcaagtttggctccgtcgtcgcctttctcctggaaaagtcgcgtgttctcacgcaggacgaacaggagcggtcgtaccacatcttctaccaaatgtgcaagggggcggacgcggcgatgaaggagcgcttccatatcctgccgctctcggagtacaagtacatcaatccgttgtgcctggacgcgccagggatcgacgacgtcgcggagttccacgaagtctgcgagtcgttccggtcgatgaatctgacggaggacgaagtcgcgagcgtgtggagcatcgtgagtggagtgctgctgcttggcaacgtcgaggtgacagcgacgaaggatggggggatcgacgacgccgcggcgatcgaggggaagaacttggaggttttcaaaaaggcctgcgggctgctcttcctcgacgcggagcgcattcgcgaagagctgacggtgaaggtttcgtatgcggggaatcaggagatccgcggccggtggaagcaggaagacggagacatgctcaagtcgtcgctcgcgaaggcgatgtacgacaagttgttcatgtggatcattgccgtgttgaaccgcagcatcaagcctccgggcggcttcaagatcttcatgggcatgctcgacatcttcggcttcgaagtcttcaagaacaactcgctggagcagttcttcatcaacatcacgaacgaaatgctgcagaagaacttcgtcgacatcgtcttcgaccgcgagagcaagctgtatcgtgacgagggtgtctcctccaaggagttgattttcacctcgaacgcagaagtgatcaagatcttgacggcgaagaacaactcggtgctcgctgcgctcgaggaccagtgcctcgcccctggaggcagcgacgaaaagttcctctcgacctgcaagaacgcgctgaaaggaaccaccaagttcaagcctgcgaaggtctctccgaacatcaatttcctcatctcgcacactgtcggcgacatccagtacaacgccgaaggcttcctcttcaaaaacaaagatgtcctgcgagcagaaatcatggaaatcgtgcagcaaagcaagaaccccgttgtcgcgcaactcttcgctggcatcgtcatggagaaggggaagatggccaagggacaactgattgggtcgcagttcctctcgcagctgcagagcctcatggaacttatcaacagcaccgagcctcacttcattcgctgcatcaagccgaacgacacgaagaagcccctcgactgggtgccgtcgaaaatgctcattcagctgcacgcgctctccgtcctcgaggctcttcagctccgtcaactcggctactcttacagacgtccgttcaaggagttcctcttccagttcaagtttatcgacctctcggcttctgaaaatccaaatctggaccccaaagaagctgcgctgagactcctcaaaagcagcaaactgcccagcgaagaataccagctcgggaagacaatggttttcctcaagcagacgggcgcgaaagaactgacgcagattcagagagaatgcctttcttcttgggagcctctcgtctcagtgctcgaggcgtactacgctggcagacgccacaagaagcagctgctgaaaaagacccccttcatcattcgcgcccaggctcacatccgcagacacctggtggacaacaacgtcagccccgcgactgttcagccggcgttcggatccactcgagatgcaggcggtgcttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 5 - protein, 864 aa, Artificial Sequence
  Description of Artificial Sequence pTetO7Sag4-MyoA
MHEQKLISEEDLLHHHHHHHDGTELASKTTSEELKTATALKKRSSDVHAVDHSGNVYKGFQIWTDLAPSVKEEPDLMFAKCIVQAGTDKGNLTCVQIDPPGFDEPFEVPQANAWNVNSLIDPMTYGDIGMLPHTNIPCVLDFLKVRFMKNQIYTTADPLVVAINPFRDLGNTTLDWIVRYRDTFDLSKLAPHVFYTARRALDNLHAVNKSQTIIVSGESGAGKTEATKQIMRYFAAAKTGSMDLRIQNAIMAANPVLEAFGNAKTIRNNNSSRFGRFMQLDVGREGGIKFGSVVAFLLEKSRVLTQDEQERSYHIFYQMCKGADAAMKERFHILPLSEYKYINPLCLDAPGIDDVAEFHEVCESFRSMNLTEDEVASVWSIVSGVLLLGNVEVTATKDGGIDDAAAIEGKNLEVFKKACGLLFLDAERIREELTVKVSYAGNQEIRGRWKQEDGDMLKSSLAKAMYDKLFMWIIAVLNRSIKPPGGFKIFMGMLDIFGFEVFKNNSLEQFFINITNEMLQKNFVDIVFDRESKLYRDEGVSSKELIFTSNAEVIKILTAKNNSVLAALEDQCLAPGGSDEKFLSTCKNALKGTTKFKPAKVSPNINFLISHTVGDIQYNAEGFLFKNKDVLRAEIMEIVQQSKNPVVAQLFAGIVMEKGKMAKGQLIGSQFLSQLQSLMELINSTEPHFIRCIKPNDTKKPLDWVPSKMLIQLHALSVLEALQLRQLGYSYRRPFKEFLFQFKFIDLSASENPNLDPKEAALRLLKSSKLPSEEYQLGKTMVFLKQTGAKELTQIQRECLSSWEPLVSVLEAYYAGRRHKKQLLKKTPFIIRAQAHIRRHLVDNNVSPATVQPAFGSTRDAGGA

SEQ ID NO: 6 - DNA, 6346 bp, Artificial Sequence
  CDS (1193)..(3787)
  misc_feature (1102): n is disclosed as an asterisk
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttcaatgtgcacctgtaggaagctgtagtcactgctgattctcactgttctcggcaagggccgacgaccggagtacagtttttgtgggcagagccgttgtgcagctttccgttcttctcggntgtgtcacatgtgtcattgtcgtgtaaacacacggttggatgtcggtttcgctgcaccacttcattatttcttctggttttttgacgagtatgcatgagcagaagctcatctccgaggaggacctgctgcatcatcatcatcatcatcatgatggtaccgagctcgcgagcaagaccacgtctgaggagctgaaaacggccacggcgctgaagaagaggtcgtccgatgtccacgcggtcgaccactccggcaatgtgtacaaaggatttcaaatctggacggacttggcgccgtcggtgaaggaggagccggacctgatgtttgccaagtgcatcgtgcaggcggggacagacaaggggaacttgacctgcgtccagatcgatccaccgggcttcgacgaaccgttcgaagtcccgcaggcgaatgcgtggaacgtaaacagcctgatcgaccccatgacgtacggagacatcggcatgttgcctcacacgaacattccttgcgtcctcgacttcctcaaggtgcgcttcatgaagaatcaaatctacacgactgcggacccgctcgtcgtcgccatcaatcccttccgcgacctcgggaacaccacgctcgactggattgttcgatacagagacactttcgacctctccaaactcgcgccccatgttttctacaccgcccgacgcgcgctcgacaacctccacgccgtcaacaagtcgcaaacgatcatcgtgtccggtgagtctggcgcgggcaagacggaggcgacgaagcagattatgaggtattttgcggcggcgaagacggggtcgatggatttgcggattcagaacgcgatcatggcggcgaatccagtgcttgaggcatttggaaatgcgaagacgattcgcaacaacaactcgtcgcgtttcggacgcttcatgcagctggatgtgggtcgcgaaggaggcatcaagtttggctccgtcgtcgcctttctcctggaaaagtcgcgtgttctcacgcaggacgaacaggagcggtcgtaccacatcttctaccaaatgtgcaagggggcggacgcggcgatgaaggagcgcttccatatcctgccgctctcggagtacaagtacatcaatccgttgtgcctggacgcgccagggatcgacgacgtcgcggagttccacgaagtctgcgagtcgttccggtcgatgaatctgacggaggacgaagtcgcgagcgtgtggagcatcgtgagtggagtgctgctgcttggcaacgtcgaggtgacagcgacgaaggatggggggatcgacgacgccgcggcgatcgaggggaagaacttggaggttttcaaaaaggcctgcgggctgctcttcctcgacgcggagcgcattcgcgaagagctgacggtgaaggtttcgtatgcggggaatcaggagatccgcggccggtggaagcaggaagacggagacatgctcaagtcgtcgctcgcgaaggcgatgtacgacaagttgttcatgtggatcattgccgtgttgaaccgcagcatcaagcctccgggcggcttcaagatcttcatgggcatgctcgacatcttcggcttcgaagtcttcaagaacaactcgctggagcagttcttcatcaacatcacgaacgaaatgctgcagaagaacttcgtcgacatcgtcttcgaccgcgagagcaagctgtatcgtgacgagggtgtctcctccaaggagttgattttcacctcgaacgcagaagtgatcaagatcttgacggcgaagaacaactcggtgctcgctgcgctcgaggaccagtgcctcgcccctggaggcagcgacgaaaagttcctctcgacctgcaagaacgcgctgaaaggaaccaccaagttcaagcctgcgaaggtctctccgaacatcaatttcctcatctcgcacactgtcggcgacatccagtacaacgccgaaggcttcctcttcaaaaacaaagatgtcctgcgagcagaaatcatggaaatcgtgcagcaaagcaagaaccccgttgtcgcgcaactcttcgctggcatcgtcatggagaaggggaagatggccaagggacaactgattgggtcgcagttcctctcgcagctgcagagcctcatggaacttatcaacagcaccgagcctcacttcattcgctgcatcaagccgaacgacacgaagaagcccctcgactgggtgccgtcgaaaatgctcattcagctgcacgcgctctccgtcctcgaggctcttcagctccgtcaactcggctactcttacagacgtccgttcaaggagttcctcttccagttcaagtttatcgacctctcggcttctgaaaatccaaatctggaccccaaagaagctgcgctgagactcctcaaaagcagcaaactgcccagcgaagaataccagctcgggaagacaatggttttcctcaagcagacgggcgcgaaagaactgacgcagattcagagagaatgcctttcttcttgggagcctctcgtctcagtgctcgaggcgtactacgctggcagacgccacaagaagcagctgctgaaaaagacccccttcatcattcgcgcccaggctcacatccgcagacacctggtggacaacaacgtcagccccgcgactgttcagccggcgttcggatccactcgagatgcaggcggtgcttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 7 - protein, 864 aa, Artificial Sequence; identical to SEQ ID NO: 5
  Description of Artificial Sequence pTetO7Sag1-MyoA

SEQ ID NO: 8 - DNA, 4556 bp, Artificial Sequence
  CDS (1270)..(2001)
  Description of Artificial Sequence pTetO7Sag4-GFP
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttacgccgctgagactaactagaaagaagtgtgcaacagttcatgagggacaaaaggaatgtgatgcggttcgcttgaagaaggaatgtttaagcacgtcaacaatacgccttggcgaatgttcatgactgttcatgtggttcatcggatcatttgaaaacatcgtgaggctggtacctggtcgcaaacgtcgtagtgtagtaccgacaataacgtcgtcgttcaaggggacgcagttctcggaagacgcgtcgcagcatactgcaactgctttcgtctgtcttcaaccatgcatagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctgcagttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 9 - protein, 243 aa, Artificial Sequence
  Description of Artificial Sequence pTetO7Sag4-GFP
MHSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYKAAVN

SEQ ID NO: 10 - DNA, 4479 bp, Artificial Sequence
  CDS (1193)..(1924)
  misc_feature (1102): n is disclosed as an asterisk
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttcaatgtgcacctgtaggaagctgtagtcactgctgattctcactgttctcggcaagggccgacgaccggagtacagtttttgtgggcagagccgttgtgcagctttccgttcttctcggntgtgtcacatgtgtcattgtcgtgtaaacacacggttggatgtcggtttcgctgcaccacttcattatttcttctggttttttgacgagtatgcatagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctgcagttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - protein, 243 aa, Artificial Sequence; identical to SEQ ID NO: 9
  Description of Artificial Sequence pTetO7Sag1-GFP

SEQ ID NO: 12 - DNA, 4438 bp, Artificial Sequence
  CDS (1193)..(1885)
  Description of Artificial Sequence pTetO7Sag1-HXGPRT
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttcaatgtgcacctgtaggaagctgtagtcactgctgattctcactgttctcggcaagggccgacgaccggagtacagtttttgtgggcagagccgttgtgcagctttccgttcttctcggttgtgtcacatgtgtcattgtcgtgtaaacacacggttgtatgtcggtttcgctgcaccacttcattatttcttctggttttttgacgagtatgcatgcgtccaaacccattgaagactacggcaagggcaagggccgtattgagcccatgtatatccccgacaacaccttctacaacgctgatgactttcttgtgcccccccactgcaagccctacattgacaaaatcctcctccctggtggattggtcaaggacagagttgagaagttggcgtatgacatccacagaacttacttcggcgaggagttgcacatcatttgcatcctgaaaggctctcgcggcttcttcaaccttctgatcgactaccttgccaccatacagaatggtcgtgagtccagcgtgccccccttcttcgagcactatgtccgcctgaagtcctaccagaacgacaacagcacaggccagctcaccgtcttgagcgacgacttgtcaatctttcgcgacaagcacgttctgattgttgaggacatcgtcgacaccggtttcaccctcaccgagttcggtgagcgcctgaaagccgtcggtcccaagtcgatgagaatcgccaccctcgtcgagaagcgcacagatcgctccaacagcttgaagggcgacttcgtcggcttcagcattgaagacgtctggatcgttggttgctgctacgacttcaacgagatgttccgcgacttcgaccacgtcgccgtcctgagcgacgccgctcgcaaaaagttcgagaaggcttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 13 - protein, 230 aa, Artificial Sequence
  Description of Artificial Sequence pTetO7Sag1-HXGPRT
MHASKPIEDYGKGKGRIEPMYIPDNTFYNADDFLVPPHCKPYIDKILLPGGLVKDRVEKLAYDIHRTYFGEELHIICILKGSRGFFNLLIDYLATIQNGRESSVPPFFEHYVRLKSYQNDNSTGQLTVLSDDLSIFRDKHVLIVEDIVDTGFTLTEFGERLKAVGPKSMRIATLVEKRTDRSNSLKGDFVGFSIEDVWIVGCCYDFNEMFRDFDHVAVLSDAARKKFEKA

SEQ ID NO: 14 - DNA, 8287 bp, Artificial Sequence
  CDS (1193)..(4318)
  Description of Artificial Sequence pTetO7Sag1-LacZ
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttcaatgtgcacctgtaggaagctgtagtcactgctgattctcactgttctcggcaagggccgacgaccggagtacagtttttgtgggcagagccgttgtgcagctttccgttcttctcggttgtgtcacatgtgtcattgtcgtgtaaacacacggttgtatgtcggtttcgctgcaccacttcattatttcttctggttttttgacgagtatgcatgccatggagaagttattattccgaagttcctattctctagaaagtataggaacttcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataattaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacggtatcgataagcttgatatgcatgtccgcgttcgtgaaattctctgatcaagcggagtgatcaccaatcatcgtctcagcgggatgacgttgcggcaagggccggctcgcggtgggcagtcagatgccgaacgtaactcaggacggcttgcgctcatcgcagaacaggggtggtgcctgcattgggtgcggttggtgatcctggttggaccggtggagatgcgcgcgcacgaaggggatgtgtcagaaacattttgtttgttctctgtgaacttttagatgtgttaaaggcggcgaatattagcagagagtcctccttgttccattctctcttgaatttcgccctttccttctctttgcgagtgtggtagagaacaagcactcgttcgccgtccctgacgacgcaacccgcgcagaagacatccaccaaacggtgttacacaatcaccttgtgtgaagttcttgcggaaaactactcgttggcattttttcttgaattccctttttcgacaaaatgcatgagaaaaaaatcactggatataccaccgttgatatatcccaatcgcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - protein, 1041 aa, Artificial Sequence
  Description of Artificial Sequence pTetO7Sag1-LacZ
MHAMEKLLFRSSYSLESIGTSSLALAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 16 - DNA, 8364 bp, Artificial Sequence
  CDS (1270)..(4395)
  Description of Artificial Sequence pTetO7Sag4-LacZ
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaggtcgacggtatcgataagcttacgccgctgagactaactagaaagaagtgtgcaacagttcatgagggacaaaaggaatgtgatgcggttcgcttgaagaaggaatgtttaagcacgtcaacaatacgccttggcgaatgttcatgactgttcatgtggttcatcggatcatttgaaaacatcgtgaggctggtacctggtcgcaaacgtcgtagtgtagtaccgacaataacgtcgtcgttcaaggggacgcagttctcggaagacgcgtcgcagcatactgcaactgctttcgtctgtcttcaaccatgcatgccatggagaagttattattccgaagttcctattctctagaaagtataggaacttcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataattaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacggtatcgataagcttgatatgcatgtccgcgttcgtgaaattctctgatcaagcggagtgatcaccaatcatcgtctcagcgggatgacgttgcggcaagggccggctcgcggtgggcagtcagatgccgaacgtaactcaggacggcttgcgctcatcgcagaacaggggtggtgcctgcattgggtgcggttggtgatcctggttggaccggtggagatgcgcgcgcacgaaggggatgtgtcagaaacattttgtttgttctctgtgaacttttagatgtgttaaaggcggcgaatattagcagagagtcctccttgttccattctctcttgaatttcgccctttccttctctttgcgagtgtggtagagaacaagcactcgttcgccgtccctgacgacgcaacccgcgcagaagacatccaccaaacggtgttacacaatcaccttgtgtgaagttcttgcggaaaactactcgttggcattttttcttgaattccctttttcgacaaaatgcatgagaaaaaaatcactggatataccaccgttgatatatcccaatcgcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - protein, 1041 aa, Artificial Sequence; identical to SEQ ID NO: 15
  Description of Artificial Sequence pTetO7Sag4-LacZ

SEQ ID NO: 18 - DNA, 8398 bp, Artificial Sequence
  CDS (1167)..(3209)
  Description of Artificial Sequence Ptub8TetR-GCN5-DHFRTS
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgacggtatcgataagcttaaccacaaaccttgagacgcgtgttccaaccacgcaccctgacacgcgtgttccaaccacgcaccctgagacgcgtgttctaaccacgcaccctgagacgcgtgttctaaccacgcaccctgagacgcgtgttcaagcttgcctgcattgggtgcggttggtgatcctggttggaccggtggagatgcgcgcgcacgaaggggatgtgtcagaaacattttgtttgttctctgtgaacttttagatgtgttaaaggcggcgaatattagcagagagtcctccttgttccattctctcttgaatttcgccctttccttctctttgcgagtgtggtagagaacaagcactcgttcgccgtccctgacgacgcaacccgcgcagaagacatccaccaaacggtgttacacaatcaccttgtgtgaagttcttgcggaaaactactcgttggcattttttcttgaattcctttttcgacaaaatgtcgcgcctggacaagagcaaagtcatcaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctgaagtgcgaaagcggtatgcataaaggcgctccaacaggtctggggctcgcgtctttcttcgggaagtcgttcatttttcacacattgcatgcggcgttgcccgcgctgttggaggaactcgccaacaccgtcgtggggacggagctgcgacgcttcgttctcgccctcgcagccgccgtcggcctctccagttcgcatgcagaggagcttctccaccgggccgtcgctgttcgttccagtcgcctcgaatcgatcttgccgtcggagacaggcttggggtttctgcaccgcgacgcgggaggagcgcgcgaagaggaactcggaatcatcagcttctgctgcgtgacgaacgacaggcaaccgttgcacatgcggcacttggtgacggtgaaaaacatcttctctcgacagctcccgaagatgccgcgagagtacatcgtccggctcgtttttgaccgagcccacttcaccttctgtctctgcaagcaaggccgcgtcatcggaggagtctgcttccgcccctacttccgcgaaaaattcgcggaaattgctttcctcgcggtgacatctactgagcaggtcaagggttacgggacgcgtctcatgaatcatctcaaggaacatgtgaagaaatctggaatcgaatatttcctcacctacgcagacaactttgcagtggggtatttccgtaagcagggcttcagcagcaagataacgatgccgcgagaccgttggttgggctacatcaaggactacgacggcggtacgttgatggagtgtcgtctcagcacccgaataaattacctgaaactttcgcagctcctcgccctacagaaactcgcagtgaagcgacgcattgagcaatccgcgccttcagtcgtctgtccttctctctctttctggaaggaaaatccaggtcagctgttgatgccgtcggccattccgggcttggccgaactaaacaagaatggcgagctgtctctgctgctgtcttcggggcgcgtcggggccgcgccccaagggtcaggggcccttcccggcgggcgcacgggcgccttgggctccaaaaagggccctttcgggcgcgcgggcttcgcgaagggcgaaaagggcctgcgcgctgcgtcactcaaggcgcagattgcggcgcttctgtcaactctggaaaagcattcttcctcttggcccttccggcgacctgtctcggtcagcgaggcccccgactactacgaggtcgtgcgaagaccgatcgacatcagcaccatgaaaaaacgaaatcgaaatggggactacagaacgaaggaggcgttccaggaagatctgctgctgatgttcgacaactgtcgcgtgtacaattcgcccgacacaatttactacaagtacgcagacgagctccaggccttcatctggcccaaggtcgaggctctcgggagtttttaattaatcaccgttgtgctcacttctcaaatcgacaaaggaaacacacttcgtgcagcatgtgccccattataaagaaactgagttgttccgctgtggcttgcaggtgtcacatccacaaaaaccggccgactctaaataggagtgtttcgcagcaagcagcgaaagtttatgactgggtccgaatctctgaacggatgtgtggcggacctggctgatgttgatcgccgtcgacacacgcgccacatgggtcaatacacaagacagctatcagttgttttagtcgaaccggttaacacaattcttgcccccccgagggggatccactagttctagagcggccgctctagaactagtggatcgatcccccgggctgcaggaattcatcctgcaagtgcatagaaggaaagttgtctgctgtcgtgggcagacagcaacagtccagcactctagcggcatacagaacgataacgcattcacgagtggatacacgcacatctgcgtcacccgcaactcgctttcgttctgattgacaaaaagaaaacaaggcgaggtgagactgtgtgaaatgccacatgaagagtcatcccttttcttcgataaaggacacaggggtctctggcaccccctcgtcagctctctccgacccgaggcactctccctgatccctccgaaaagagaggaaaacgagagacgggcagcttctgtatttccgctagacagccatctccatctggattcgtccgtgcgggacgtagcccacgacctcaaaatcctcggcggtgaaatcgtcgatttccttgatgcgttccttgttgaggatgttcacaatggggaacggtctcggttctctccgcagctgctcttttaaagcctcgacatggttcgtgtagacatgcgtgttccccatgaagtgaatgaactccttaggttttaggttgcagacgtgtgcaaccatgagcgtcaaaagcgaataggaagcgatgttgaaggggacgccgaggccgacatcgcacgaccgctgatacatgatgcacgacagctccttctggtcgttcacgtagaactggcacaacaagtgacaaggcggcagcgccatttcgtccagcgctgcaggattccaggcagtcatgagcatgcgacgatctgttggattcgttctcagcatctggatcacattcttcagctggtcgacgccctgccctgtgtagtctgtgtgcatgtctttgtatgccgcgccgaagtgtctccactggaagccgtagcccgggccgatgtctccgacctctcggtgggggagattgcgcgaatcgaggaactcgcgtgtcacattcttgtcccagatcttcacgcccttctcagaaagatggtttgcgttcgtgtcgccgcgaatgaaccacagcaactcttcgaggacccctttccagaacacacgctttgtggtgagaagtggaaaggcctgatccagcgagtagcgcatagtgcagccgaatttggagatgacaccaacgcccgttcggtcatccattgtccttccattgttaataatgtcggcaatgagatcaaggtactggaattcttcatggcctctaaagtgaacatgcggaacggcccgaatcagttccttttgctcgcgttttttccggtcttcttcgtccatccacgccaacaccggggcaatggctgcggccgaagaaggagcctgcaacccgtgcacgggagttgtctccctcgtggacgtcaaggagctcattgcgttgctcggttccgcagtggctgcgtcgtcagtcttccttctcttctcgagaaccacaaagtcgtagggtaccccgttgtctgagaaggtcttggaaatgaagatgggtcgatacgtcgcttcattgtccttctctcttccgagctccggacaaaagggaacgaacacagactcggcaggagctgcagcctgcgcagcagttgatttgtttgaaagaatgtcatctccggggaacgcagggaagaaaacgtcgcacggaaactcgcgggctacacgcgtgatgtacaggtgagaggcaacgcccagagacagcgctgcctcgtacagtcccgctcctcccacgacaaaaatctggtcgacagaatccttgtactcttcctccagaaggctgagagctgctgggagtgaagcacagactcggacgcgctgctggccttcagcttgaggcttctccgccgcaatgtcttcttctttgagggaagaggaaacgacgatgttcaatctgtccacgaggggtctaaactttcgaggcatgctttcccaggttttccgtcccatgacaacggcgttgaatctcttgccgactgatggagagggaagtccagagtcgcccgtctttgcaaatttcctgggaagccacccgttcaggcgactggcttcttcgggcgtcgtttttgtcacacgagaaaagtgtttgaaatctgtggtcaagtggggccacgggaggccgttgttgatgccgatgcccctcttgggggtcatcgcgacgaccagacacaccggtttctgcatcttcccagacacgacaacgccccgtagagcagaaacgcactactaaagcgaaacttcacccgtccctgctgcactcagagcagtgctccgcactgccgtgtggtaaaatgaaaaggttctacgagacacgcgtctccggatcgacaagcgaaggatctgcacacctggtctcgatgtcgaacaaagcacggaggagagacggaaagtgcttacatcgaacacggttatcaaacccgagaaaaagaaacgaacagaagaaaaaggaaacctccgcatacttttaaagaatgaagttccccgattttcccaaaaatggcgtcattttcgcgcacggcagtcagataacaggtgtagcggctgcccaccaacagagacggcgcggccgacaggacgctactgggactgcgaacagcagcaagatcggatcttccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 19 - protein, 680 aa, Artificial Sequence
  Description of Artificial Sequence Ptub8TetR-GCN5-DHFRTS
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGMHKGAPTGLGLASFFGKSFIFHTLHAALPALLEELANTVVGTELRRFVLALAAAVGLSSSHAEELLHRAVAVRSSRLESILPSETGLGFLHRDAGGAREEELGIISFCCVTNDRQPLHMRHLVTVKNIFSRQLPKMPREYIVRLVFDRAHFTFCLCKQGRVIGGVCFRPYFREKFAEIAFLAVTSTEQVKGYGTRLMNHLKEHVKKSGIEYFLTYADNFAVGYFRKQGFSSKITMPRDRWLGYIKDYDGGTLMECRLSTRINYLKLSQLLALQKLAVKRRIEQSAPSVVCPSLSFWKENPGQLLMPSAIPGLAELNKNGELSLLLSSGRVGAAPQGSGALPGGRTGALGSKKGPFGRAGFAKGEKGLRAASLKAQIAALLSTLEKHSSSWPFRRPVSVSEAPDYYEVVRRPIDISTMKKRNRNGDYRTKEAFQEDLLLMFDNCRVYNSPDTIYYKYADELQAFIWPKVEALGSF

SEQ ID NO: 20 - DNA, 6216 bp, Artificial Sequence
  CDS (1167)..(1844)
  Description of Artificial Sequence pTub8TATi-1-HXGPRT
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgacggtatcgataagcttaaccacaaaccttgagacgcgtgttccaaccacgcaccctgacacgcgtgttccaaccacgcaccctgagacgcgtgttctaaccacgcaccctgagacgcgtgttctaaccacgcaccctgagacgcgtgttcaagcttgcctgcattgggtgcggttggtgatcctggttggaccggtggagatgcgcgcgcacgaaggggatgtgtcagaaacattttgtttgttctctgtgaacttttagatgtgttaaaggcggcgaatattagcagagagtcctccttgttccattctctcttgaatttcgccctttccttctctttgcgagtgtggtagagaacaagcactcgttcgccgtccctgacgacgcaacccgcgcagaagacatccaccaaacggtgttacacaatcaccttgtgtgaagttcttgcggaaaactactcgttggcattttttcttgaattcctttttcgacaaaatgtcgcgcctggacaagagcaaagtcatcaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacccacgttctttaatagtggactcttgttccaaactggaacaacactcaaccctatctcggtctattcttttgatttataagggattttgccgatttcggcctattggttaaaaaatgagttgatttaccaaaaatttaacgcgaattttaacaaaatattaacgcttacaatttaggtggcccttttcggggaaatgtgcgcggaacccctatttgtttatttttttaaatacattcaaatatgtatccgctcatgagccaataaccctgataaatgcttcaataatatcaaaaaaaaaaaaaaaaaaaaaaaaaaaaaaaaaaaaaggatccactagttctagagcggccgccaccgcggtgtcactgtagcctgccagaacacttgtcaaccgactgtgtccacatttttatgcgcactgactggcatgaatggccagaggcaggcatcagcaagtcacgtaggccaacgcgtgcgcagaaacgctcaaggctcgattgtgggtgggggttggtagcattttatcgacctaaacaaggtttacacttaggtggtgcggttttactgatctggacggattcagcggtcgcagattatcgatctgcaaatggtgtacacttaggtgtcgcggcttatttagttaagggagcttcgtggtcggagcctaacaagtcaacagagacgtatcgccaatcgttcgcggtgaagagtcgaaactgacagcacatcgtagggaaactgagagggtgctcctttctctccgtcgtttgcgctgcaccatcctgcaagtgcatagaaggaaagttgtctgctgtcgtgggcagacagcaacagtccagcactctagcggcatacagaacgataacgcattcacgagtggatacacgcacatctgcgtcacccgcaactcgctttcgttctgattgacaaaaagaaaacaaggcgaggtgagactgtgtgaaatgccacatgaagagtcatcccttttcttcgataaaggacacaggggtctctggcaccccctcgtcagctctctccgacccgaggcactctccctgatccctccgaaaagagaggaaaacgagagacgggcagcttctgtagggctatgcagggtttacttctcgaactttttgcgagcggcgtcgctcaggacggcgacgtggtcgaagtcgcggaacatctcgttgaagtcgtagcagcaaccaacgatccagacgtcttcaatgctgaagccgacgaagtcgcccttcaagctgttggagcgatctgtgcgcttctcgacgagggtggcgattctcatcgacttgggaccgacggctttcaggcgctcaccgaactcggtgagggtgaaaccggtgtcgacgatgtcctcaacaatcagaacgtgcttgtcgcgaaagattgacaagtcgtcgctcaagacggtgagctggcctgtgctgttgtcgttctggtaggacttcaggcggacatagtgctcgaagaaggggggcacgctggactcacgaccactgtacttctgtatggtggcaaggtagtcgatcagaaggttgaagaagccgcgagagcctttcaggatgcaaatgatgtgcaactcctcgccgaagtaagttctgtggatgtcatacgccaacttctcaactctgtccttgaccaatccaccagggaggaggattttgtcaatgtagggcttgcagtgggggggcacaagaaagtcatcagcgttgtagaaggtgttgtcggggatatacatgggctcaatacggcccttgcccttgccgtagtcttcaatgggtttggacgccattttggatctgacaacgccccgtagagcagaaacgcactactaaagcgaaacttcacccgtccctgctgcactcagagcagtgctccgcactgccgtgtggtaaaatgaaaaggttctacgagacacgcgtctccggatcgacaagcgaaggatctgcacacctggtctcgatgtcgaacaaagcacggaggagagacggaaagtgcttacatcgaacacggttatcaaacccgagaaaaagaaacgaacagaagaaaaaggaaacctccgcatacttttaaagaatgaagttccccgattttcccaaaaatggcgtcattttcgcgcacggcagtcagataacaggtgtagcggctgcccaccaacagagacggcgcggccgacaggacgctactgggactgcgaacagcagcaagatcggatcttccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 21 - protein, 225 aa, Artificial Sequence
  Description of Artificial Sequence pTub8TATi-1-HXGPRT
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKPTFFNSGLLFQTGTTLNPISVYSFDL

SEQ ID NO: 22 - DNA, 6392 bp, Artificial Sequence
  CDS (1167)..(1823)
  Description of Artificial Sequence pTub8TATi-3-HXGPRT
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgacggtatcgataagcttaaccacaaaccttgagacgcgtgttccaaccacgcaccctgacacgcgtgttccaaccacgcaccctgagacgcgtgttctaaccacgcaccctgagacgcgtgttctaaccacgcaccctgagacgcgtgttcaagcttgcctgcattgggtgcggttggtgatcctggttggaccggtggagatgcgcgcgcacgaaggggatgtgtcagaaacattttgtttgttctctgtgaacttttagatgtgttaaaggcggcgaatattagcagagagtcctccttgttccattctctcttgaatttcgccctttccttctctttgcgagtgtggtagagaacaagcactcgttcgccgtccctgacgacgcaacccgcgcagaagacatccaccaaacggtgttacacaatcaccttgtgtgaagttcttgcggaaaactactcgttggcattttttcttgaattcctttttcgacaaaatgtcgcgcctggacaagagcaaagtcatcaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctgactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcaccgagtgggttacatcgaactggatctcaacagcggtaagatccttgagagttttcccacccaaaaacacccccaaacacaaaaaaaaacaaaggatccactagttctagagcggccgccaccgcggtgtcactgtagcctgccagaacacttgtcaaccgactgtgtccacatttttatgcgcactgactggcatgaatggccagaggcaggcatcagcaagtcacgtaggccaacgcgtgcgcagaaacgctcaaggctcgattgtgggtgggggttggtagcattttatcgacctaaacaaggtttacacttaggtggtgcggttttactgatctggacggattcagcggtcgcagattatcgatctgcaaatggtgtacacttaggtgtcgcggcttatttagttaagggagcttcgtggtcggagcctaacaagtcaacagagacgtatcgccaatcgttcgcggtgaagagtcgaaactgacagcacatcgtagggaaactgagagggtgctcctttctctccgtcgtttgcgctgcaccatcctgcaagtgcatagaaggaaagttgtctgctgtcgtgggcagacagcaacagtccagcactctagcggcatacagaacgataacgcattcacgagtggatacacgcacatctgcgtcacccgcaactcgctttcgttctgattgacaaaaagaaaacaaggcgaggtgagactgtgtgaaatgccacatgaagagtcatcccttttcttcgataaaggacacaggggtctctggcaccccctcgtcagctctctccgacccgaggcactctccctgatccctccgaaaagagaggaaaacgagagacgggcagcttctgtagggctatgcagggtttacttctcgaactttttgcgagcggcgtcgctcaggacggcgacgtggtcgaagtcgcggaacatctcgttgaagtcgtagcagcaaccaacgatccagacgtcttcaatgctgaagccgacgaagtcgcccttcaagctgttggagcgatctgtgcgcttctcgacgagggtggcgattctcatcgacttgggaccgacggctttcaggcgctcaccgaactcggtgagggtgaaaccggtgtcgacgatgtcctcaacaatcagaacgtgcttgtcgcgaaagattgacaagtcgtcgctcaagacggtgagctggcctgtgctgttgtcgttctggtaggacttcaggcggacatagtgctcgaagaaggggggcacgctggactcacgaccactgtacttctgtatggtggcaaggtagtcgatcagaaggttgaagaagccgcgagagcctttcaggatgcaaatgatgtgcaactcctcgccgaagtaagttctgtggatgtcatacgccaacttctcaactctgtccttgaccaatccaccagggaggaggattttgtcaatgtagggcttgcagtgggggggcacaagaaagtcatcagcgttgtagaaggtgttgtcggggatatacatgggctcaatacggcccttgcccttgccgtagtcttcaatgggtttggacgccattttggatctgacaacgccccgtagagcagaaacgcactactaaagcgaaacttcacccgtccctgctgcactcagagcagtgctccgcactgccgtgtggtaaaatgaaaaggttctacgagacacgcgtctccggatcgacaagcgaaggatctgcacacctggtctcgatgtcgaacaaagcacggaggagagacggaaagtgcttacatcgaacacggttatcaaacccgagaaaaagaaacgaacagaagaaaaaggaaacctccgcatacttttaaagaatgaagttccccgattttcccaaaaatggcgtcattttcgcgcacggcagtcagataacaggtgtagcggctgcccaccaacagagacggcgcggccgacaggacgctactgggactgcgaacagcagcaagatcggatcttccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 23 - protein, 218 aa, Artificial Sequence
  Description of Artificial Sequence pTub8TATi-3-HXGPRT
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLTLVPNWNNTQPYLGLFF

SEQ ID NO: 24 - DNA, 77 bp, Artificial Sequence
  Description of Artificial Sequence TATi-1 nucleotide sequence of activating domain
cccacgttctttaatagtggactcttgttccaaactggaacaacactcaaccctatctcggtctattcttttgattt

SEQ ID NO: 25 - protein, 26 aa, Artificial Sequence
  Description of Artificial Sequence TATi-1 presumed amino acid sequence of activating domain
PTFFNSGGLLFQTTTLNPISVYSFDL

SEQ ID NO: 26 - protein, 224 aa, Artificial Sequence
  Description of Artificial Sequence TATi-1
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKPTFFNSGLLFQTGTTLNPISVYSFD

SEQ ID NO: 27 - DNA, 57 bp, Artificial Sequence
  Description of Artificial Sequence TATi-3 nucleotide sequence of activating domain
cagctgactcttgttccaaactggaacaacactcaaccctatctcggtctattcttt

SEQ ID NO: 28 - protein, 19 aa, Artificial Sequence
  Description of Artificial Sequence TATi-3 presumed amino acid sequence of activating domain
QLTLVPNWNNTQPYLGLFF

SEQ ID NO: 29 - protein, 218 aa, Artificial Sequence; identical to SEQ ID NO: 23
  Description of Artificial Sequence TATi-3

SEQ ID NO: 30 - DNA, 58 bp, Artificial Sequence
  Description of Artificial Sequence Oligonucleotide
cggaattccttttcgacaaaatgtcgcgcctggacaagagcaaagtcatcaactctgc

SEQ ID NO: 31 - DNA, 37 bp, Artificial Sequence
  Description of Artificial Sequence Oligonucleotide
cccttaattaatgcataccgctttcgcacttcagctg